(19) United States
(12) Patent Application Publication
(10) Pub. No.: US 2006/0121055 A1
(43) Pub. Date: Jun. 8, 2006

(54) Compositions with enhanced immunogenicity

(75) Inventors: Robert L. Campbell, Bahama, NC (US); Kevin Dolan, Holly Springs, NC (US); Jason Alarcon, Durham, NC (US); John Mikszta, Durham, NC (US); Wendy Woodley, Cary, NC (US); Sheetal Mehta, East Windsor, NJ (US)

Correspondence Address: JONES DAY, 222 EAST 41ST ST, NEW YORK, NY, 10017, US

(73) Assignee: Becton, Dickinson and Company, Inc.

(21) Appl. No.: 11/006,086

(22) Filed: Dec. 6, 2004

(51) Int. Cl. A61K 39/145; A61K 31/716

(52) U.S. Cl. 424/209.1; 514/57

(57) ABSTRACT

The present invention relates to immunogenic compositions containing an immunogen and a specific combination of two or more traditional excipients. The excipients in the composition act in combination and enhance immune responses to the immunogen from a subject. The combination of excipients may be used as adjuvant in immunogenic compositions, regardless of route or target of delivery. The compositions can be administered, for example, intradermally, epidermally, transdermally, junctionally, nasally, or subcutaneously.

### 1. FIELD OF THE INVENTION

The present invention relates to immunogenic compositions, designed to provide an enhanced immunogenicity from the immunogen encompassed in such compositions. The immunogenic compositions of the invention comprise an antigenic or immunogenic agent, and two or more excipients, which, in combination with one another, enhance the immunogenicity of the antigenic or immunogenic agent, resulting in an enhanced immune response. Any route of delivery, such as intradermal, transdermal, intramuscular, epidermal, junctional, and subcutaneous, can be used in connection with the immunogenic compositions of the invention. The enhanced efficacy of the immunogenic compositions results in a therapeutically effective immune response after a single dose, with lower doses of antigenic or immunogenic agent than conventionally used, and without the need for booster immunizations.

### 2. BACKGROUND OF THE INVENTION

2.1 Excipients

Pharmaceutical dosage forms contain both active ingredients, and inactive ingredients called excipients. The behavior of the dosage form is dependent on process variables and the interrelationship between the various excipients and their impact on the active ingredient. Excipients are therefore employed to effect various characteristics that improve the behavior of the dosage form to achieve better efficacy. For example, excipients are used in a pharmaceutical formulation to achieve higher stability, better resistance to biological or chemical deterioration, higher solubility, and/or reduced surface tension for ease of delivery. Conventional excipients differ from adjuvants in that they are not known to directly enhance the efficay of the active ingredient, for example, immunogenicity of an antigen or immunogen in an immunogenic formulation.

2.2 Vaccines

Vaccines have traditionally consisted of live attenuated pathogens, whole inactivated organisms or inactivated toxins. In many cases these approaches have been successful at inducing immune protection based on antibody mediated responses. However, certain pathogens, e.g., HIV, HCV, TB, and malaria, require the induction of cell-mediated immunity (CMI). Non-live vaccines have generally proven ineffective in producing CMI. In addition, although live vaccines may induce CMI, some live attenuated vaccines may cause disease in immunosuppressed subjects. As a result of these problems, several new approaches to vaccine development have emerged, such as recombinant protein subunits, synthetic peptides, protein polysaccharide conjugates, and plasmid DNA. While these new approaches may offer important safety advantages, a general problem is that vaccines alone are often poorly immunogenic. Therefore, there is a continuing need for the development of potent and safe adjuvants that can be used in vaccine formulations to enhance their immunogenicity. For a review of the state of the art in vaccine development see, e.g., Edelman, 2002, Molecular Biotech. 21: 129-148; O'Hagan et al., 2001, Biomolecular Engineering, 18: 69-85; Singh et al., 2002, Pharm. Res. 19(6):715-28)

Traditionally, the immunogenicity of a vaccine formulation has been improved by injecting it in a formulation that includes an adjuvant. Immunological adjuvants were initially described by Ramon (1924, Ann. Inst. Pasteur, 38:1) “as substances used in combination with a specific antigen that produced a more robust immune response than the antigen alone”. A wide variety of substances, both biological and synthetic, have been used as adjuvants. However, despite extensive evaluation of a large number of candidates over many years, the only adjuvants currently approved by the U.S. Food and Drug administration are aluminum-based minerals (generically called Alum). Alum has a debatable safety record (see, e.g., Malakoff, Science, 2000, 288: 1323), and comparative studies show that it is a weak adjuvant for antibody induction to protein subunits and a poor adjuvant for CMI. Moreover, Alum adjuvants can induce IgE antibody response and have been associated with allergic reactions in some subjects (see, e.g., Gupta et al., 1998, Drug Deliv. Rev. 32: 155-72; Relyveld et al., 1998, Vaccine 16: 1016-23). Many experimental adjuvants have advanced to clinical trials since the development of Alum, and some have demonstrated high potency but have proven too toxic for therapeutic use in humans. Thus, an on-going need exists for safe and potent adjuvants.

The existing vaccine formulations are usually administered several times over a time span of months in order to elicit an immune response that can confer protection on the host upon subsequent encounter with the antigen, e.g., microbe, itself. Thus, although vaccines for a variety of infectious diseases are currently available, many of these, including those for influenza, tetanus, and hepatitis B, require more than one administration to confer a protective benefit. These limitations are extremely problematic in countries where healthcare is not readily available or accessible. Moreover, compliance is also a problem in developed countries, particularly for childhood immunization programs.

Therefore, there is clearly an unmet need for more effective vaccine formulations to result in an enhanced therapeutic efficacy and protective immune response. There is also a need to develop vaccine formulations that reduce or eliminate the need for prolonged injection regimens.

### 3. SUMMARY OF THE INVENTION

The present invention is based, in part, on the surprising discovery by the inventors that delivering an antigenic or immunogenic agent in combination with two or more pre-selected excipients results in an enhanced immune response to the antigenic or immunogenic agent. The enhanced efficacy of the compositions of the invention are based, in part, on the appreciation and recognition by the inventors that specific combinations of pre-selected excipients can act as adjuvants, resulting in an enhanced immune response to an antigenic or immunegic agent.

The benefits of the invention are also appreciated in all compartments, including, but not limited to, intradermal, epidermal, intramuscular, transdermal, junctional, and subcutaneous compartments. Without being limited by a particular theory, it is found that a combination of two or more pre-selected excipients can synergistically or additively act to enhance the immunogenicity of the antigen or immunogen comprised in the compositions of the invention, resulting in a better immune response to the antigen or immunogen.

The immunogenic compositions of the invention comprise a combination of two or more pre-selected excipients. In one embodiment, the composition of the invention comprises lutrol in combination with one or more other excipients. Examples of other excipients include, but are not limited to, methylcellulose, gelatin, sorbitol, chitosan, and urea. In another embodiment, the composition of the invention comprises methylcellulose in combination with one or more other excipients. Examples of other excipients include, but are not limited to, lutrol, gelatin, sorbitol, chitosan, and urea. In another embodiment, the composition of the invention comprises gelatin in combination with one or more other excipients. Examples of other excipients include, but are not limited to, lutrol, methylcellulose, sorbitol, chitosan, and urea. In another embodiment, the composition of the invention comprises sorbitol in combination with one or more other excipients. Examples of other excipients include, but are not limited to, lutrol, methylcellulose, gelatin, chitosan, and urea. In another embodiment, the composition of the invention comprises chitosan in combination with one or more other excipients. Examples of other excipients include, but are not limited to, lutrol, methylcellulose, gelatin, sorbitol, and urea. In another embodiment, the composition of the invention comprises urea in combination with one or more other excipients. Examples of other excipients include, but are not limited to, lutrol, methylcellulose, gelatin, sorbitol, and chitosan.

Excipients which may be used in the immunogenic compositions of the invention include, but are not limited to, stabilizers, preservatives, solvents, surfactants or detergents, suspending agents, tonicity agents, geling agents, muco/bioadhesives, vehicles and ingredients for growth medium. A non-limiting list of excipients that may be used in the immunogenic compositions of the invention are acetic acid, citric acid, fumaric acid, hydrochloric acid, nitric acid, sodium acetate, cellulose, charcoal, gelatin, ammonia solution, ammonium carbonate, mono-, di- or tri-ethanolamine, potassium hydroxide, sodium borate, sodium carbonate, sodium hydroxide, trolamine, nitrogen gas, ascorbic acid, ascorbyl palmitate, butylated hydroxyanisole, butylated hydroxytoluene, hypophosphorous acid, monothioglycerol, propyl gallate, sodium ascorbate, sodium bisulfite, sodium formaldehyde sulfoxylate, sodium metabisulfite, sodium sulfite, glycine, potassium metaphosphate, potassium phosphate, monobasic sodium acetate, anhydrous or dihydrate sodium citrate, edetate disodium, edetic acid, glycerin, propylene glycol and sorbitol, amphotericin B, benzoic acid, methyl-, ethyl-, propyl- or butyl-paraben, sodium benzoate and sodium propionate, amiprilose, benzalkonium chloride, benzethonium chloride, benzyl alcohol, betapropiolactone, cetylpyridium chloride, chlorobutanol, chlortetracycline, EDTA, formaldehyde, gentamicin, kanamycin, neomycin, phenol, phenoxyethanol, phenylethyl alcohol, phenylmercuric nitrate, polymyxin B, streptomycin, thimerosal, tri-(n)-butyl phosphate, nystatin, water, alcohol especially ethyl alcohol, corn oil, cottonseed oil, glycerin, isopropyl alcohol, mineral oil, oleic acid, peanut oil, purified water, water for injection, sterile water for injection, benzalkonium chloride, magnesium stearate, nonoxynol 10, oxtoxynol 9 (Triton N-101), pluronic or poloxamers such as pluronic F-127, pluronic F-68, pluronic F-108, poloxamer 124, 188 (Lutrol F-68), 237, 388 or 407 (Lutrol F-127), polysorbate 20 (Tween™ 20), polysorbate 80 (Tween™ 80), sodium lauryl sulfate, sorbitan monopalmitate, agar, bentonite, carbomers (e.g., Carbopols such as carbopol EX55), carboxymethylcellulose sodium, gelatin, hydroxyethyl cellulose, hydroxypropyl cellulose, hydroxypropyl methylcellulose, kaolin, methylcellulose, tragacanth, veegum, carboxymethylcellulose sodium, gelatin, dextrose, glucose, sodium chloride, corn oil, mineral oil, peanut oil, sesame oil, bacteriostatic sodium chloride, bacteriostatic water, amino acids, bactopeptone, bovine albumin, bovine serum, egg protein, human serum albumin, mouse serum proteins, MRC-5 cellular protein, ovalbumin, vitamins, yeast proteins, apo-transferrin, aprotinin, anti-foaming agents such as polydimethylsilozone, silicon, fetuin (a serum protein), glycolic acid (a skin exfoliate), hydrogen peroxide (a detoxifier), lactose (a filler), mannose, urea, polycarbophils, polyacrylic acid (PAA), capricol, hyaluronic acid, chitosans, lectins, sodium alginate, pectin, acacia, and povidone.

Antigenic or immunogenic agents that may be used in the immunogenic compositions of the invention include antigens from an animal, a plant, a bacteria, a protozoan, a parasite, a virus or a combination thereof. The antigenic or immunogenic agent may be any viral peptide, protein, polypeptide, or a fragment thereof derived from a virus including, but not limited to, RSV-viral proteins, e.g., RSV F glycoprotein, RSV G glycoprotein, influenza viral proteins, e.g., influenza virus neuramimidase, influenza virus hemagglutinin, herpes simplex viral protein, e.g., herpes simplex virus glycoprotein including for example, gB, gC, gD, and gE. The antigenic or immunogenic agent for use in the compositions of the invention may be an antigen of a pathogenic virus such as, an antigen of adenovirdiae (e.g., mastadenovirus and aviadenovirus), herpesviridae (e.g., herpes simplex virus 1, herpes simplex virus 2, herpes simplex virus 5, and herpes simplex virus 6), leviviridae (e.g., levivirus, enterobacteria phase MS2, allolevirus), poxyiridae (e.g., chordopoxyirinae, parapoxvirus, avipoxvirus, capripoxvirus, leporipoxvirus, suipoxvirus, molluscipoxvirus, and entomopoxyirinae), papovaviridae (e.g., polyomavirus and papillomavirus), paramyxoviridae (e.g., paramyxovirus, parainfluenza virus 1, mobillivirus (e.g., measles virus), rubulavirus (e.g., mumps virus), pneumonovirinae (e.g., pneumovirus, human respiratory syncytial virus), metapneumovirus (e.g., avian pneumovirus and human metapneumovirus), picornaviridae (e.g., enterovirus, rhinovirus, hepatovirus (e.g., human hepatitis A virus), cardiovirus, and apthovirus), reoviridae (e.g., orthoreovirus, orbivirus, rotavirus, cypovirus, fijivirus, phytoreovirus, and oryzavirus), retroviridae (e.g., mammalian type B retroviruses, mammalian type C retroviruses, avian type C retroviruses, type D retrovirus group, BLV-HTLV retroviruses), lentivirus (e.g. human immunodeficiency virus 1 and human immunodeficiency virus 2), spumavirus, flaviviridae (e.g., hepatitis C virus), hepadnaviridae (e.g., hepatitis B virus), togaviridae (e.g., alphavirus (e.g., sindbis virus) and rubivirus (e.g., rubella virus), rhabdoviridae (e.g., vesiculovirus, lyssavirus, ephemerovirus, cytorhabdovirus, and necleorhabdovirus), arenaviridae (e.g., arenavirus, lymphocytic choriomeningitis virus, Ippy virus, and lassa virus), and coronaviridae (e.g., coronavirus and torovirus).

Alternatively, the antigenic or immunogenic agent in the immunogenic compositions of the invention may be a cancer or tumor antigen including but not limited to, KS ¼ pan-carcinoma antigen, ovarian carcinoma antigen (CA125), prostatic acid phosphate, prostate specific antigen, melanoma-associated antigen p97, melanoma antigen gp75, high molecular weight melanoma antigen (HMW-MAA), prostate specific membrane antigen, carcinoembryonic antigen (CEA), polymorphic epithelial mucin antigen, human milk fat globule antigen, colorectal tumor-associated antigens such as: CEA, TAG-72, CO17-1A; GICA 19-9, CTA-1 and LEA, Burkitt's lymphoma antigen-38.13, CD19, human B-lymphoma antigen-CD20, CD33, melanoma specific antigens such as ganglioside GD2, ganglioside GD3, ganglioside GM2, ganglioside GM3, tumor-specific transplantation type of cell-surface antigen (TSTA) such as virally-induced tumor antigens including T-antigen DNA tumor viruses and Envelope antigens of RNA tumor viruses, oncofetal antigen-alpha-fetoprotein such as CEA of colon, bladder tumor oncofetal antigen, differentiation antigen such as human lung carcinoma antigen L6, L20, antigens of fibrosarcoma, human leukemia T cell antigen-Gp37, neoglycoprotein, sphingolipids, breast cancer antigen such as EGFR (Epidermal growth factor receptor), HER2 antigen (p185^{HER2}), polymorphic epithelial mucin (PEM), malignant human lymphocyte antigen-APO-1, differentiation antigen such as I antigen found in fetal erythrocytes, primary endoderm, I antigen found in adult erythrocytes, preimplantation embryos, I(Ma) found in gastric adenocarcinomas, M18, M39 found in breast epithelium, SSEA-1 found in myeloid cells, VEP8, VEP9, Myl, VIM-D5, D₁56-22 found in colorectal cancer, TRA-1-85 (blood group H), C14 found in colonic adenocarcinoma, F3 found in lung adenocarcinoma, AH6 found in gastric cancer, Y hapten, Le^{y} found in embryonal carcinoma cells, TL5 (blood group A), EGF receptor found in A431 cells, E₁ series (blood group B) found in pancreatic cancer, FC10.2 found in embryonal carcinoma cells, gastric adenocarcinoma antigen, CO-514 (blood group Le^{a}) found in Adenocarcinoma, NS-10 found in adenocarcinomas, CO-43 (blood group Le^{b}), G49 found in EGF receptor of A431 cells, MH2 (blood group ALe^{b}/Le^{y}) found in colonic adenocarcinoma, 19.9 found in colon cancer, gastric cancer mucins, T₅A₇ found in myeloid cells, R₂₄ found in melanoma, 4.2, G_{D3}, D1.1, OFA-1, G_{M2}, OFA-2, G_{D2}, and M1:22:25:8 found in embryonal carcinoma cells, and SSEA-3 and SSEA-4 found in 4 to 8-cell stage embryos, and T cell receptor derived peptide from a Cutaneous T cell Lymphoma.

The antigenic or immunogenic agent for use in the immunogenic compositions of the invention may be any substance that under appropriate conditions results in an immune response in a subject, including, but not limited to, polypeptides, peptides, proteins, glycoproteins, lipids, nucleic acids and polysaccharides. The concentration of the antigenic or immunogenic agent in the immunogenic compositions of the invention may be determined using standard methods known to one skilled in the art and depends on the potency and nature of the antigenic or immunogenic agent. Given the enhanced efficay provided by the compositions of the invention, the concentration of the antigenic or immunogenic agent is preferably less than the conventional amounts used.

The immunogenic compositions of the invention are particularly advantageous for developing rapid and high levels of immunity against the antigenic or immunogenic agent, against which an immune response is desired. The immunogenic compositions of the invention can achieve a systemic immunity at a protective level with a low dose of the antigenic or immunogenic agent. In some embodiments, the compositions of the invention result in a protective immune response with a dose of the antigenic or immunogenic agent which is 80%, 60%, 50%, or 40% of the dose conventionally used for the antigenic or immunogenic agent in obtaining an effective immune response. In preferred embodiments, the compositions of the invention comprise a dose of the antigenic or immunogenic agent which is lower than the conventional dose used in the art, e.g., the dose recommended in the Physician's Desk Reference. Preferably, the compositions of the invention result in a therapeutically or prophylactically effective immune response after a single dose.

The immunogenic compositions of the instant invention have an enhanced therapeutic efficacy, safety, and toxicity profile relative to currently available formulations. The benefits and advantages imparted by the compositions of the invention is, in part, due to the particular formulation, i.e., synergistic or additive combinations of two or more excipients. Preferably, the compositions of the invention provide a greater and more durable protection, especially for high risk populations that do not respond well to immunization.

Without being limited by a particular theory, the therapeutic efficacy of the vaccine formulations of the invention is, in part, due to the ability of the combination of excipients to allow the exposure of the antigenic or immunogenic agent to the immune cells of the tissue, by recruiting antigen presenting cells to the site of injection, resulting in an enhanced immune response to the antigenic or immunogenic agent. Furthermore, without being limited by a particular theory, when the combination of excipients is administered at the concentrations and by the delivery routes in accordance with the methods of the invention, it may exhibit non-specific adjuvant activity, i.e., not through a specific cellular receptor, but perhaps through promotion of mechanical damage, mild irritation, or stretching of the tissue compartment. In one embodiment, the immunogenic compositions of the invention are therapeutically and/or prophylactically effective in enhancing the immune response in an immumologically immature, suppressed or senescent subject.

The invention further contemplates kits comprising an immunogenic composition of the invention, along with the device/reagents necessary for specific routes of delivery contemplated. In a specific embodiment, the invention provides a kit comprising, one or more containers filled with one or more of the components of the immunogenic compositions of the invention, e.g., an antigenic or immunogenic agent or an excipient. In another specific embodiment, the kit comprises two or more containers, one containing an antigenic or immunogenic agent, and the rest containing the one or more excipients, or combinations thereof. Associated with such container(s) can be a notice in the form prescribed by a governmental agency regulating the manufacture, use or sale of pharmaceuticals or biological products, which notice reflects approval by the agency of manufacture, use or sale for human administration.

3.1 Definitions

As used herein, and unless otherwise specified, the term “excipient” means an ingredient or an additive in a composition, which itself possesses no pharmacological or biological activity for which the composition is intended, and preferably which, prior to the instant invention, was not known to directly enhance or otherwise alter such pharmacological or biological activity when administered to a subject, particularly in combination with one or more other excipients. Excipients used in the methods of the present invention are pre-selected excipients. As used herein, “pre-selected” excipients encompass traditional, non-traditional, and any other exicipient that has an adjuvant activity when delivered to a subject in accordance with the methods described herein.

As used herein, a “traditional” excipient is a more or less inert substance added in a composition as a diluent or vehicle. Alternatively, a traditional excipient may be used to give form or consistency to a composition. Examples of such traditional excipients are known to one skilled in the art and encompassed within the instant invention, see, e.g., Remington's Pharmaceutical Sciences, Mack Pub. Co., N.J., current edition; all of which is incorporated herein by reference in its entirety.

As used herein a “traditional” adjuvant is a substance added to a composition to enhance the antigenicity of the active ingredient in the composition, e.g., a suspension of minerals, on which an antigenic or immunogenic agent is absorbed, or water-in-oil emulsion in which an antigenic agent is emulsified in mineral oil (e.g., Freunds incomplete adjuvant), sometimes with the inclusion of killed mycobacteria to further enhance the antigenicity of the antigenic agent.

### 4. BRIEF DESCRIPTION OF THE FIGURES

FIG. 1. Rats (n=10 per group) were immunized intramuscularly with trivalent Fluzone® vaccine alone or reformulated with 5% lutrol and 0.18% methylcellulose. Sera were collected on d21 and screened for antibodies specific to the H1N1 New Caledonia strain or the H3N2 Panama strain by HAI assay.

FIG. 2. Guinea pigs (n=10 per group) were immunized with trivalent Fluzone® vaccine alone intramuscularly or intradermally, or intradermally with Fluzone® vaccine reformulated with 5% lutrol and 0.18% methylcellulose. Sera were collected on d21 and screened against a cocktail consisting of H1N1 New Calcdonia strain, the H3N2 Panama strain and the Hong Kong B strain by HAI assay.

FIG. 3 Mice (n=10 per group) were immunized intramuscularly or intradermally with trivalent Fluzone® vaccine alone or intradermally with Fluzone® vaccine reformulated with 15% lutrol and 0.18% methylcellulose. Sera were collected on d21 and screened for antibodies specific to the H1N1 New Calcdonia strain or the H3N2 Panama strain by HAI assay.

FIG. 4. Mice (n=10 per group) were immunized intramuscularly or intradermally with trivalent Fluzone® vaccine alone or intradermally with Fluzone® vaccine reformulated with 5% lutrol and 0.18% methylcellulose. Sera were collected on d21 and screened for antibodies specific to the H1N1 New Caledonia strain or the H3N2 Panama strain by HAI assay.

FIG. 5. Guinea pigs (n=10 per group) were immunized intramuscularly or intradermally with trivalent Fluzone® vaccine alone or intradermally with Fluzone® vaccine reformulated with 5% lutrol and 0.18% methylcellulose. Sera were collected on d21 and screened for antibodies specific to the H3N2 Panama strain by HAI assay.

FIG. 6. Guinea pigs (n=10 per group) were immunized with trivalent Fluzone® vaccine alone intramuscularly or intradermally with Fluzone® vaccine reformulated with 5% lutrol and 0.2% urea. Sera were collected on d21 and screened against a cocktail consisting of H1N1 New Calcdonia strain, the H3N2 Panama strain and the Hong Kong B strain by HAI assay.

FIG. 7. Guinea pigs (n=10 per group) were immunized intramuscularly with trivalent Fluzone® vaccine alone or intradermally with Fluzone® vaccine reformulated with 5% lutrol and 0.2% urea. Sera were collected on d21 and screened for antibodies specific to the H1N1 New Calcdonia strain, the H3N2 Panama strain or the Hong Kong B strain by HAI assay.

FIG. 8 Guinea pigs (n=10 per group) were immunized with trivalent Fluzone® vaccine alone intramuscularly, or intradermally or intradermally with Fluzone® vaccine reformulated with 0.225% gelatin and 0.18% methylcellulose. Sera were collected on d21 and screened against a cocktail consisting of H1N1 New Calcdonia strain, the H3N2 Panama strain and the Hong Kong B strain by HAI assay.

FIG. 9. Guinea pigs (n=10 per group) were immunized with trivalent Fluzone® vaccine alone intramuscularly, or intradermally or intradermally with Fluzone® vaccine reformulated with 5% Lutrol and 5% D-Sorbitol. Sera were collected on d21 and screened against a cocktail consisting of H1N1 New Calcdonia strain, the H3N2 Panama strain and the Hong Kong B strain by HAI assay.

### 5. DETAILED DESCRIPTION OF THE INVENTION

5.1 Immunogenic Compositions

The immunogenic compositions of the invention are designed to elicit an enhanced immunogenicity from the antigenic or immunogenic agent, regardless of the route or site of delivery. The immunogenic compositions of the invention comprise an antigenic or immunogenic agent and at least two excipients, which, in combination, enhance the presentation and/or availability of the antigenic or immunogenic to an immune cell, resulting in an enhanced immune response.

In one embodiment, the immunogenic composition of the invention comprises lutrol in combination with one or more other excipients. The concentration of lutrol used in the composition of the invention in combination with other excipients may be from about 0.001% w/v to about 50% w/v, from about 0.01% w/v to about 45% w/v, from about 1% w/v to about 40% w/v, from about 2% w/v to about 30% w/v, from about 3% w/v to about 20% w/v, from about 5% w/v to about 15% w/v, from about 5% w/v to about 10% w/v, or from about 3% w/v to about 7% w/v.

In one embodiment, the immunogenic composition of the invention comprises methylcellulose in combination with one or more other excipients. The concentration of methylcellulose used in the composition of the invention in combination with other excipients may be from about 0.0001% w/v to about 20% w/v, from about 0.001% w/v to about 15% w/v, from about 0.005% w/v to about 10% w/v, from about 0.01% w/v to about 5% w/v, from about 0.05% w/v to about 2% w/v, from about 0.001% w/v to about 1% w/v, from about 0.005% w/v to about 0.5% w/v, or from about 0.01% w/v to about 0.1% w/v.

In another embdoiment, the immunogenic composition of the invention comprises gelatin in combination with one or more other excipients. The concentration of gelatin used in the composition of the invention may be from about 0.001 w/v to about 30% w/v, from about 0.005% w/v to about 20% w/v, from about 0.01% w/v to about 10% w/v, from about 0.01% w/v to about 5% w/v, from about 0.01% w/v to about 0.5% w/v, from about 0.05 w/v to about 3% w/v, or from about 0.1% w/v to about 0.3% w/v.

In one embodiment, the immunogenic composition of the invention comprises sorbitol in combination with one or more other excipients. The concentration of sorbitol used in the composition of the invention in combination with other excipients may be from about 0.001% w/v to about 50% w/v, from about 0.01% w/v to about 45% w/v, from about 1% w/v to about 40% w/v, from about 2% w/v to about 30% w/v, from about 3% w/v to about 20% w/v, from about 5% w/v to about 15% w/v, from about 5% w/v to about 10% w/v, or from about 3% w/v to about 7% w/v.

In one embodiment, the immunogenic composition of the invention comprises chitosan in combination with one or more other excipients. The concentration of chitosan used in the composition of the invention in combination with other excipients may be from about 0.001% w/v to about 30% w/v, from about 0.005% w/v to about 20% w/v, from about 0.01% w/v to about 10% w/v, from about 0.01% w/v to about 5% w/v, from about 0.05% w/v to about 1% w/v, from about 0.05% w/v to about 3% w/v, or from about 0.1% w/v to about 0.5% w/v.

In one embodiment, the immunogenic composition of the invention comprises urea in combination with one or more other excipients. The concentration of urea used in the composition of the invention in combination with other excipients may be from about 0.001% w/v to about 50% w/v, from about 0.005% w/v to about 40% w/v, from about 0.01% w/v to about 30% w/v, from about 0.05% w/v to about 20% w/v, from about 0.1% w/v to about 10% w/v, from about 1% w/v to about 15% w/v, from about 0.1% w/v to about 5% w/v, or from about 0.2% w/v to about 2% w/v.

In one specific embodiment, an immunogenic composition of the invention comprises the combination of lutrol and methylcellulose. The concentration of lutrol used in the immunogenic compositions of the invention may be from about 1% w/v to about 25% w/v, from about 3% w/v to about 15% w/v, or from about 5% w/v to about 10% w/v. The concentration of methylcellulose used in the immunogenic compositions of the invention may be from about 0.001% w/v to about 1% w/v, from about 0.01% w/v to about 0.5% w/v, or from about 0.02% w/v to about 0.1% w/v.

In another embodiment, an immunogenic composition of the invention comprises the combination of lutrol and sorbitol. The concentration of lutrol used in the immunogenic compositions of the invention may be from about 1% w/v to about 25% w/v, from about 3% w/v to about 15% w/v, or from about 5% w/v to about 10% w/v. The concentration of sorbitol used in the immunogenic compositions of the invention may be from about 0.5% w/v to about 25% w/v, from about 3% w/v to about 15% w/v, or from about 5% w/v to about 10% w/v.

In another embodiment, an immunogenic composition of the invention comprises the combination of lutrol and and urea. The concentration of lutrol used in the immunogenic compositions of the invention may be from about 1% w/v to about 25% w/v, from about 3% w/v to about 15% w/v, or from about 5% w/v to about 10% w/v. The concentration of urea used in the immunogenic compositions of the invention may be from about 0.01% w/v to about 40% w/v, from about 0.1% w/v to about 10% w/v, or from about 0.2% w/v to about 1% w/v.

In another embodiment, an immunogenic composition of the invention comprises the cobmination of lutrol and chitosan. The concentration of lutrol used in the immunogenic compositions of the invention may be from about 1% w/v to about 25% w/v, from about 3% w/v to about 15% w/v, or from about 5% w/v to about 10% w/v. The concentration of chitosan used in the immunogenic composition of the invention may be from about 0.01% w/v to about 1% w/v, from about 0.05% w/v to about 0.5% w/v, or from about 0.1% w/v to about 0.25% w/v.

In another embodiment, an immunogenic composition of the invention comprises the combination of methylcellulose and gelatin. The concentration of methylcellulose used in the immunogenic compositions of the invention may be from about 0.001% w/v to about 1% w/v, from about 0.01% w/v to about 0.5% w/v, or from about 0.02% w/v to about 0.1% w/v. The concentration of gelatin used in the immunogenic composition of the invention may be from about 0.01% w/v to about 5% w/v, from about 0.05% w/v to about 0.5% w/v, or from about 0.1% w/v to about 0.225 w/v.

In another embodiment, an immunogenic composition of the invention comprises the combination of lutrol and gelatin. The concentration of lutrol used in the immunogenic compositions of the invention may be from about 1% w/v to about 25% w/v, from about 3% w/v to about 15% w/v, or from about 5% w/v to about 10% w/v. The concentration of gelatin used in the immunogenic compositions of the invention may be from about 0.01% w/v to about 5% w/v, from about 0.05% w/v to about 0.5% w/v, or from about 0.1% w/v to about 0.225 w/v.

Although not intending to be bound by a particular mechanism of action, the immunogenic compositions of the invention achieve an enhanced therapeutic efficacy, e.g., enhanced protective immune response, in part, due to the persistance of the antigenic or immunogenic agent at the site of the injection, i.e., the “depot effect.” Preferably, the immunogenic compositions of the invention decrease the clearance rate of the antigenic or immunogenic agent from the site of the injection. More preferably, the immunogenic compositions of the invention allow slow release of the antigenic or immunogenic agent at the site of injection. In a specific embodiment, the molecule acts to prolong the exposure of the antigenic or immunogenic agent to the immune cells, e.g., antigen presenting cells and/or Langerhan's cells (LC), resulting in an enhanced protective immune response. Alternatively, the immunogenic compositions of the invention have enhanced efficacy, e.g., enhanced protective immune response, as the antigenic or immunogenic agent is delivered to patients, with an enhanced availability and/or presentation to the immune cells, e.g., antigen presenting cells. The enhanced efficacy of the compositions of the invention results in a therapeutically effective response, e.g., protective immune response, after a single dose, with lower doses of the antigenic or immunogenic agent than conventionally used, and without the need for booster immunizations.

Furthermore, without being bound by a particular mechanism of action, the immunogenic compositions of the invention may enhance the immunological response or therapeutic efficacy of the antigenic or immunogenic agent by (1) enhancing the immunogenicity of the antigenic or immunogenic agent; (2) enhancing the speed and/or duration of the immune response; (3) modulating the avidity, specificity, isotype or class distribution of the antibody response; (4) stimulating cell-mediated immune response; (5) promoting mucosal immunity; or (6) decreasing the dose of the antigenic or immunogenic agent.

Although not intending to be bound by a particular mode of action, the immunogenic compositions of the invention enhance cell-mediated immune response by specifically targeting the antigenic or immunogenic agent to the antigen presenting cells, e.g., dendritic cells and Langerhan cells. The immunogenic compositions of the invention may enhance cell-mediated and/or humoral mediated immune response. Cell-mediated immune responses that may be modulated by the vaccine formulations of the invention include for example, Th1 or Th2 CD4+ T-helper cell-mediated or CD8+ cytotoxic T-lymphocytes mediates responses.

Excipients that may be used, in combination with one or more of the other, in the immunogenic compositions of this invention include, but are not limited to, stabilizers, preservatives, solvents, surfactants or detergents, suspending agents, tonicity agents, vehicles and ingredients for growth medium. Examples of excipients that may be used in the compositions and methods of the invention are disclosed herein in Section 5.2.1 and exemplified in Examples. The concentration of the excipient used in the immunogenic compositions of the invention depends on the particular excipient used. In some embodiments, the concentration of the excipient used in the immunogenic compositions of the invention may be at 0.000002% to 58% (w/v) and 0.05% to 0.45% (v/v). In other embodiments, the concentration of the excipient used may be at least 10% (w/v), at least 15% (w/v), at least 20% (w/v), at least 25% (w/v), or at least 30% (w/v). In other embodiments, the concentration of the excipient is greater than about 30% (w/v). In yet other embodiments, the concentration of the excipient is at least 0.1% (w/v), at least 0.5% (w/v), at least 1% (w/v), at least 5% (w/v), or at least 10% (w/v).

Excipients may be used in the preparation and manufacturing of immunogenic compositions. In such cases, residual concentrations of the excipient may be found in the final immunogenic composition, left over from the manufacturing or preparation of the composition. Such residual concentrations are too low to result in the adjuvant activity observed with the immunogenic compositions of the invention.

Other molecules which may be used in the immunogenic compositions of the invention include geling agents such as polymers that polymerize or gel, e.g., form a semi-solid or solid two or three dimensional matrix. Preferably, such molecules once administered to the tissue, thus allow, for example, interaction and exposure of the antigenic or immunogenic agent with the immunological space therein. In most preferred embodiments, polymers used in the compositions of the invention do not form liposomal or micellar structures. The polymer preferably enhances the presentation and/or availability of the antigenic or immunogenic agent to the immune cells. Preferably, the molecule used in the immunogenic composition of the invention is biocompatible and/or biodegradable. In a specific embodiment, the molecule is a biomolecule, including, but not limited to, a protein, a polypeptide, and a peptide.

In some embodiments, the molecule used in the immunogenic compositions of the invention is any polymer that undergoes a physical transition from a liquid to a gel at a physiological temperature of the subject to which the composition is administered, e.g., in the case of a human subject, at a temperature ranging from 25° to 37° C. In some embodiments, the physical transition does not comprise a liposome or a micelle. Preferably, the liquid to gel transition of the polymer used in the immunogenic compositions of the invention is thermally induced, and most preferably is reversible. In some embodiments, the liquid-gel transition of the polymer is chemically induced. The liquid-gel transition temperature of the polymer is preferably below the physiological temperature of the subject to which the immunogenic composition is administered. In some embodiments, the transition of the polymer from a liquid to a gel also results in an increase in the viscosity of the polymer, by at least 30%, at least 50%, at least 60%, at least 80%, at least 90%, or at least 99%. In preferred embodiments, the polymer is a non-ionic block copolymer, including, but not limited to, Pluronic F-127, Pluronic F-108, and Pluronic F108. The polymer may have one or more characteristics of an adjuvant, a bioadhesive, or a mucoadhesive.

One advantage of the use of polymers in the compositions of the invention is that, at a temperature below the physiological temperature, e.g., a temperature ranging from 25° to 37° C., the composition is a liquid, and after the injection, the composition forms a gel as it is warmed in the subject to a temperature above the liquid-gel transition temperature. In a specific embodiment, the gelatinous formulation may allow slow release of the antigenic or immunogenic agent in the tissue, potentiating an effective immune response. The ease of delivery of the composition is another advantage since the gelatinous material prevents any fluid leakage.

Other molecules which may be used in the immunogenic compositions of the invention are bio or mucoadhesives, which are advantageous, in part, since they may allow the antigenic or immunogenic agent to adhere to the biological and immunological surface of the tissue space. A non-limiting example of bio or mucoadhesive that may be used in the immunogenic compositions of the invention are, polycarbophils, capricol, polyacrylic acid (PAA), carobopols, Carbopol EX55, carbomers, polysaccharides, hyaluronic acid, chitosans; lectins; cellulose, methylcellulose, carboxymethylcellulose, hydroxypropyl methyl cellulose, sodium alginate, gelatin, pectin, acacia, and povidone.

The concentration of these other moelcules in the immunogenic compositions of the invention depends on the particular molecule used. In a specific embodiment, when the molecule is a polymer the concentration of the polymer used in the immunogenic compostions of the invention may be at least 5% (w/v), at least 10% (w/v), at least 15% (w/v), at least 20% (w/v), at least 25% (w/v), or at least 30% (w/v). In some embodiments, the concentration of the polymer is greater than about 30% (w/v). In another specific embodiment, when the molecule is a muco or bioadhesive, the concentration used in the immunogenic compositions of the invention may be at least 0.1% (w/v), at least 0.5% (w/v), at least 1% (w/v), at least 5% (w/v), or at least 10% (w/v).

In some embodiments, the immunogenic compositions of the invention comprise one or more additives including, but not limited to, a traditional adjuvant, a traditional excipient, a stabilizer, and a penetration enhancer. A traditional excipient, is a more or less inert substance added in a composition as a diluent or vehicle. Alternatively, a traditional excipient may be used to give form or consistency to a composition. Examples of such traditional excipients are known to one skilled in the art and encompassed within the instant invention, see, e.g., Remington's Pharmaceutical Sciences Mack Pub. Co., N.J., current edition; all of which is incorporated herein by reference in its entirety. A traditional adjuvant, is a substance added to a composition to enhance the antigenicity of the active ingredient in the composition, e.g., a suspension of minerals, on which an antigenic or immunogenic agent is absorbed, or water-in-oil emulsion in which an antigenic agent is emulsified in mineral oil (e.g., Freunds incomplete adjuvant) sometimes with the inclusion of killed mycobacteria to further enhance the antigenicity of the antigenic agent.

In other embodiments, the immunogenic compositions of the present invention may further comprise one or more other pharmaceutically acceptable carriers, including any suitable diluent or excipient. Preferably, the pharmaceutically acceptable carrier does not itself induce a physiological response, e.g., an immune response. Most preferably, the pharmaceutically acceptable carrier does not result in any adverse or undesired side effects and/or does not result in undue toxicity. Pharmaceutically acceptable carriers for use in the immunogenic compositions of the invention include, but are not limited to, saline, buffered saline, dextrose, water, glycerol, sterile isotonic aqueous buffer, and combinations thereof. Additional examples of pharmaceutically acceptable carriers, diluents, and excipients are provided in Remington's Pharmaceutical Sciences (Mack Pub. Co., N.J., current edition; all of which is incorporated herein by reference in its entirety).

In particular embodiments, the immunogenic compositions of the invention, may also contain wetting agents, emulsifying agents, or pH buffering agents. The immunogenic compositions of the invention can be a solid, such as a lyophilized powder suitable for reconstitution, a liquid solution, a suspension, a tablet, a pill, a capsule, a sustained release formulation, or a powder.

The immunogenic compositions of the invention may be in any form suitable for delivery to a patient. In one embodiment, the immunogenic composition of the invention is in the form of a flowable, injectable medium, i.e., a low viscosity formulation that may be injected in a syringe. In another embodiment, the immunogenic composition of the invention is in the form of a gelatinous matrix, e.g., a semi-solid or solid two or three dimensional matrix. In yet another embodiment, the immunogenic composition of the invention is in the form of a highly viscous, thick medium with limited fluidity. In either embodiment, the antigenic or immunogenic agent is uniformly and homogenously dispersed throughout the formulation. In another embodiment, the immunogenic composition is capable of transitioning from a flowable, injectable medium to a gel, and vice versa, by a change in temperature so that the composition is in the form of a flowable, injectable medium below the transition temperature and a gel above the transition temperature. The flowable, injectible medium may be a liquid. Alternatively, the flowable, injectable medium is a liquid in which particulate material is suspended, such that the medium retains fluidity to be injectable and syringible, e.g., can be administered using a syringe.

Preferably, the immunogenic compositions of the invention are stable formulations, i.e., undergo minimal to no detectable level of degradation and/or aggregation of the antigentic or immunogenic agent, and can be stored for an extended period of time with no loss in biological activity, e.g., antigenicity or immunogenicity of the antigenic agent. In some embodiments, the stability of the immunogenic composition of the invention is, in part, due to the antigenic or immuonogenic agent being embedded, e.g., uniformly and homogeneously dispersed, in the gelatinous matrix of the polymer, which provides a stable polymeric structural network that protects and shields the antigenic or immunogenic agent from degradation and/or other unwanted modifications that result in a decrease in biological activity.

In some embodiments, the immunogenic compositions of the present invention exhibit stability at the temperature ranges of 2° C.-8° C., preferably at 4° C., for at least 2 years, as assessed by high performance size exclusion chromatography (HPSEC). Namely, the immunogenic compositions of the present invention have low to undetectable levels of aggregation and/or degradation of the anitgenic or immunogenic agent, after the storage for the defined periods as set forth above. Preferably, no more than 5%, no more than 4%, no more than 3%, no more than 2%, no more than 1%, and most preferably no more than 0.5%, of the antigenic or immunogenic molecule forms an aggregate or degrades as measured by HPSEC, after the storage for the defined periods as set forth above. Furthermore, the immunogenic compositions of the present invention exhibit almost no loss in biological activity of the antigenic or immunogenic agent during the prolonged storage under the conditions described above, as assessed by standard methods known in the art. The immunogenic compositions of the present invention retain after the storage for the above-defined periods more than 80%, more than 85%, more than 90%, more than 95%, more than 98%, more than 99%, or more than 99.5% of the initial biological activity prior to the storage.

The concentration of the antigenic or immunogenic agent in the immunogenic compositions of the invention may be determined using standard methods skilled in the art, and depends on the potency and nature of the antigenic or immunogenic agent. Given the enhanced immunogenicity provided by the compositions of the invention, the concentration of the antigenic or immunogenic agent is preferably less than the conventional amounts used. The concentration of the antigenic or immunogenic agent used in the immunogenic compositions of the invention is 90%, 80%, 60%, 50%, or 40% of the concentration conventionally used in obtaining an effective immune response. Typically, the starting concentration of the antigenic or immunogenic agent in the immunogenic composition of the invention is the amount that is conventionally used for eliciting the desired immune response. The concentration of the antigenic or immunogenic agent in the immunogenic compositions of the invention is then adjusted, e.g., by dilution using a suitable diluent, so that an effective protective immune response is achieved, as assessed using standard methods known in the art and described herein.

In some embodiments, the components of the immunogenic compositions of the invention, e.g., the antigenic or immunogenic agent, excipients, and the polymer, are supplied either separately or mixed together in unit dosage form, for example, as a dry lyophilized powder or water free concentrate in a hermetically sealed container such as an ampoule or a sachette indicating the quantity of the active agent, e.g., the antigenic or immunogenic agent. In other embodiments, an ampoule of sterile diluent can be provided so that the components may be mixed prior to administration. In a specific embodiment, the excipients and/or the polymer may be mixed with the antigenic or immunogenic agent just prior to administration. In another specific embodiment, the excipients and/or the polymer may be mixed with the antigenic or immunogenic agent in a delivery device during administration.

The invention also provides immunogenic compositions that are packaged in a hermetically sealed container such as an ampoule or a sachette indicating the quantity of the components. In one embodiment, the immunogenic composition is supplied as a liquid, in another embodiment, as a dry sterilized lyophilized powder or water free concentrate in a hermetically sealed container and can be reconstituted, e.g., with water or saline to the appropriate concentration for administration to a subject.

In an alternative embodiment, the immunogenic composition is supplied in liquid form in a hermetically sealed container indicating the quantity and concentration of the components.

The immunogenic composition of the invention may be prepared by any method that results in a stable, sterile, injectable formulation. In a specific embodiment, when a polymer is included in the composition, the polymer may be dissolved in an aqueous solution, e.g., water, at a temperature below the liquid-gel transition temperature of the polymer and at a concentration such that above the liquid-gel transition temperature a gelatinous matrix may be formed. The optimal concentration at which the polymer solution is formed depends on the particular polymer and is discussed below in Section 5.2.3. In the same embodiment, the antigenic or immunogenic agent is dissolved in an aqueous solution, e.g., water, and combined with the polymer such that a stable, sterile, injectable formulation is formed. Alternatively, the antigenic or immunogenic agent may be particulate and dissolved in the polymeric solution such that a stable, sterile, injectable formulation is formed. For enhanced performance of the immunogenic composition of the invention, the antigenic or immunogenic agent should be uniformly dispersed throughout the gelatinous matrix, which can be achieved by dissolving the antigenic or immunogenic agent in a solution comprising the polymer at a temperature below the liquid-gel transition temperature of the polymer so that once the temperature is raised the antigenic or immunogenic agent is uniformly dispersed and embedded in the gelatinous matrix.

The invention also provides a pharmaceutical pack or kit comprising an immunogenic composition of the invention. In a specific embodiment the invention provides a kit comprising, one or more containers filled with one or more of the components of the immunogenic composition of the invention, e.g., an anitgenic or immunogenic agent, two or more excipients, and other optional components. In another specific embodiment, the kit comprises two or more containers, one containing an anitgenic or immunogenic agent, and the others containing the excipients and/or other optional components. Associated with such container(s) can be a notice in the form prescribed by a governmental agency regulating the manufacture, use or sale of pharmaceuticals or biological products, which notice reflects approval by the agency of manufacture, use or sale for human administration.

The invention encompasses a method for immunization and/or stimulating an immunological immune response in a subject comprising delivering a single dose of an immunogenic composition of the invention to a subject, preferably a human. In some embodiments, the invention encompasses one or more booster immunizations.

5.2 Components

5.2.1 Excipients

The invention is based, in part, on the unexpected discovery by the inventors that delivering an antigenic or immunogenic agent in combination with a combination of two or more excipients results in an enhanced immune response to the antigenic or immunogenic agent. As used herein, and unless otherwise specified, the term “excipient” means an ingredient or an additive in a pharmaceutical composition, which itself possesses no pharmacological or biological activity for which the composition is intended. Excipients used in the methods of the present invention are pre-selected excipients. As used herein, “pre-selected” excipients encompass traditional, non-traditional, and any other exicipient that, in combination with one another, has an adjuvant activity when delivered to a patient. It has been unexpectedly discovered that specific combinations of two or more of these excipients, when co-administered with an antigenic or immunogenic agent, act as an adjuvant, i.e., enhance the immune response to the antigenic or immunogenic agent in a subject receiving such composition as compared to a subject receiving the composition without the combination of excipients.

In some embodiments, without being bound by a particular mechanism of action, when the combination of excipients of the instant invention is administered at the concentrations and by the delivery routes in accordance with the methods of the invention, they may exhibit non-specific adjuvant activity, perhaps through promotion of mechanical damage, mild irritation, or stretching of the skin. In some embodiments, without being bound by a particular mechanism of action, once the combination of excipients are delivered to a subject in accordance with the present invention, they may act as a skin irritant leading to the recruitment of antigen presenting cells at the site of the injection, and thus act as an adjuvant, i.e., enhance the immune response to the immunogenic composition.

As used herein, when the excipients as an irritant, they cause a reversible and asymptomatic inflammatory effect on tissue by chemical action at the site of contact and yet is not corrosive. Inflammatory effect at the site of injection involves an influx of blood at the site of injection and may be marked by swelling, redness, heat, and/or pain. One skilled in the art can determine if an excipient is a skin irritant using, for example, the methods disclosed in Code of Federal Regulation (Title 16, Vol. 2; 6 CFR 1500.41, which is incorporated herein by reference in its entirety). According to 6 CFR 1500.41, a chemical is a skin irritant if, when tested on the intact skin of albino rabbits by the methods of 16 CFR 1500.41 for four hours exposure or by other appropriate techniques, it results in an empirical score of five or more. Preferably, the excipients used in the methods of the invention have a score of 5 or less, more preferably a score of 4 or less, and most preferably a score of 3 or less. When an excipient of the invention is characterized as a skin irritant, one or more other excipients that are not skin irritants may be used in the immunogenic compositions to reduce the skin irritation. In a specific embodiment, in order to determine if the immunogenic composition of the invention results in skin irritation, once the immunogenic composition, e.g., a vaccine, is delivered to a subject, e.g., an animal, the site of the injection is visually checked within one hour of the immunization, at 24 hours and again at 21 days. Any observation other than the initial “Bleb” which resolves in hours, would be noted as unacceptable. In a specific embodiment, when a DNA immunogenic agent, e.g., pDNA-HA is delivered to a subject, the site of the injection is checked within one hour of the immunization (prime or boost), 24 hours afterwards, at 21 days just before boost, 24 hours after the boost and 21 days after the boost (actual day 42 of schedule).

Excipients are typically classified into subclasses according to their function. Excipients used in the immunogenic compositions of the invention may have one or more functions. Several subclasses of excipients are known in the art and are encompassed in the present invention. See, e.g., Ansel et al., Pharmaceutical Dosage Forms and Drug Delivery System, 6^{th} Ed., pp. 110-133, Williams & Wilkins (1995), which is incorporated herein by reference in its entirety. For example, an excipient can be categorized as a stabilizer, a preservative, a solvent, a surfactant or detergent, a suspending agent, a tonicity agent or a vehicle. In the case of vaccines, ingredients for growth medium, which are used to facilitate or maintain the growth of the immunogen, are commonly used as excipients. Some excipients have more than one function and can be used for multiple purposes. It will be apparent to those of ordinary skill in the art that these subclasses are not an exhaustive list of all available excipients, thus other types of excipients can also be used in accordance with the immunogenic compositions and methods of the invention. Additional categories and examples of excipients are provided in Handbook of Pharmaceutical Excipients, 2003 (4^{th} ed., American Pharmaceutical Association, London), the entirety of which is incorporated herein by reference.

In one embodiment, at least one of the excipients used in the immunogenic compositions of the invention is a stabilizer. As used herein, a stabilizer is a chemical agent that increases the stability of a pharmaceutical composition. As used herein, a stable composition refers to a composition that undergoes minimal to no detectable level of degradation and/or aggregation of the antigenic or immunogenic agent, and can be stored for an extended period of time with no loss in biological activity, e.g., antigenicity or immunogenicity of the antigenic agent. Preferably, the immunogenic compositions of the present invention exhibit stability at the temperature ranges of 2° C.-8° C., preferably at 4° C., for at least 2 years, as assessed by high performance size exclusion chromatography (HPSEC). Preferably, the immunogenic compositions of the present invention to have low to undetectable levels of aggregation and/or degradation of the antigenic or immunogenic agent, after the storage for the defined periods as set forth above. Preferably, no more than 20%, no more than 10%, no more than 5%, no more than 4%, no more than 3%, no more than 2%, no more than 1%, and most preferably no more than 0.5%, of the antigenic or immunogenic molecule forms an aggregate or degrades as measured by HPSEC, after the storage for the defined periods as set forth above. In most preferred embodiments, the immunogenic compositions of the present invention will exhibit almost no loss in biological activity of the antigenic or immunogenic agent during a prolonged storage under the conditions described above, as assessed by standard methods known in the art. The immunogenic compositions of the present invention retain after the storage for the above-defined periods more than 80%, more than 85%, more than 90%, more than 95%, more than 98%, more than 99%, or more than 99.5% of the initial biological activity prior to the storage.

Depending on the mechanism by which an excipient stabilizes the composition, the stabilizers can be further categorized into an acidifying or alkalinizing agent, an adsorbent, an air displacement agent, an antioxidant, a buffering agent, a chelating agent or a humectant, which are all encompassed within the instant invention. An acidifying agent as used herein stabilizes a pharmaceutical composition by providing an acidic medium for the active ingredient in the composition, i.e., the antigenic or immunogenic agent, that is otherwise labile in an alkaline condition. Examples of an acidifying agent include, but are not limited to, acetic acid, citric acid, fumaric acid, hydrochloric acid, nitric acid and sodium acetate. An alkalinizing agent stabilizes the composition by providing an alkaline medium for the active ingredient in the composition, i.e., the antigenic or immunogenic agent that are labile in an acidic environment. Examples of an alkalinizing agent include, but are not limited to, ammonia solution, ammonium carbonate, mono-, di- or tri-ethanolamine, potassium hydroxide, sodium borate, sodium carbonate, sodium hydroxide and trolamine.

In a specific embodiment, at least one of the excipients used in the immunogenic composition of the invention is an adsorbent. An adsorbent as used herein is an agent capable of allowing other molecules to adhere or adsorb onto its surface by physical and/or chemical means. Examples of an adsorbent include, but are not limited to, cellulose, charcoal and gelatin.

In another embodiment, at least one of the excipients used in an immunogenic composition of the invention is an air displacement agent. An air displacement agent as known to one skilled in the art is employed to displace air in a hermetically sealed container to enhance the stability of a pharmaceutical composition. Examples include, but are not limited to, nitrogen gas.

In another embodiment, at least one of the excipients used in an immunogenic composition of the invention is an antioxidant. Although not intending to be bound by a particular mechanism of action an antioxidant stabilizes a pharmaceutical composition by inhibiting oxidation, and thus preventing the deterioration of the composition by the oxidative process. Examples of an antioxidant for use in the immunogenic compositions of the invention include, but are not limited to, ascorbic acid, ascorbyl palmitate, butylated hydroxyanisole, butylated hydroxytoluene, hypophosphorous acid, monothioglycerol, propyl gallate, sodium ascorbate, sodium bisulfite, sodium formaldehyde sulfoxylate, sodium metabisulfite and sodium sulfite.

In another embodiment, at least one of the excipients used in an immunogenic composition of the invention is a buffering agent. Although not intending to be bound by a particular mechanism of action, a buffering agent stabilizes a pharmaceutical composition by providing resistance to alterations in pH for example, upon dilution or addition of acid or alkali. Examples of buffering agents that may be used in the immunogenic compositions of the invention include, but are not limited to, glycine, potassium metaphosphate, potassium phosphate, monobasic sodium acetate, and anhydrous or dihydrate sodium citrate.

In another embodiment, at least one of the excipient used in an immunogenic composition of the invention is a chelating agent. Although not intending to be bound by a particular mechanism of action, a chelating agent stabilizes a pharmaceutical composition by forming a stable, water soluble complex with one or more metals, e.g., heavy metals. Heavy metals are typically critical in enzymatic activity of proteases, and thus chelating agents limit the activity of the proteases by sequestering a metal needed for their enzymatic activity. Examples of a chelating agents that may be used in the compositions of the invention include, but are not limited to, edetate disodium and edetic acid.

In another embodiment, at least one of the excipients used in an immunogenic compositions of the invention is a humectant. A humectant is an agent that prevents the drying out of preparations by retaining moisture. Examples of humectants that may be used in the immunogenic compositions of the invention include, but are not limited to, glycerin, propylene glycol and sorbitol. In a specific embodiment, at least one the excipients of this invention is sorbitol. Preferably, the concentration of sorbitol used in the immunogenic compositions of the invention may be from about 0.5% w/v to about 25% w/v, from about 3% w/v to about 15% w/v, or from about 5% w/v to about 10% w/v.

In another embodiment, at least one of the excipientss used in an immunogenic composition of this invention is a preservative. Although not intending to be bound by a particular mechanism of action a preservative is a substance that prevents the growth of exogenous organisms in a pharmaceutical composition. Preservatives include, for example, antifungal agents, i.e., an agent that prevents the growth of fungi, and antimicrobial agents, i.e., an agent that prevents the growth of microorganisms including viruses. Examples of antifungal agents that may be used in the immunogenic compositions and methods of the invention include, but are not limited to, amphotericin B, benzoic acid, methyl-, ethyl-, propyl- or butyl-paraben, sodium benzoate and sodium propionate. Examples of antimicrobial agents that may be used in the immunogenic compositions and methods of the invention include, but are not limited to, amiprilose, benzalkonium chloride, benzethonium chloride, benzyl alcohol, betapropiolactone, cetylpyridium chloride, chlorobutanol, chlortetracycline, EDTA, formaldehyde, gentamicin, kanamycin, neomycin, phenol, phenoxyethanol, phenylethyl alcohol, phenylmercuric nitrate, polymyxin B, streptomycin, thimerosal, tri-(n)-butyl phosphate.

In another embodiment, at least one of the excipients used in an immunogenic compositions of the invention is a solvent. Examples of solvents include, but are not limited to, ethanol.

In another embodiment, at least one of the excipients used in an immunogenic composition of the invention is a surfactant, i.e., surface active agent. Although not intending to be bound by a particular mechanism of action a surfactant absorbs to a surface or an interface and reduces surface or interfacial tension. A surfactant may be used as a wetting agent, detergent or emulsifying agent. Examples of a surfactants that may be used in the compositions of the invention include, but are not limited to, benzalkonium chloride, magnesium stearate, nonoxynol 10, oxtoxynol 9 (Triton N-101), poloxamers such as poloxamer 124, 188 (Lutrol F 68), 237, 388 or 407 (Lutrol F 127), polysorbate 20 (Tween 20), polysorbate 80 (Tween 80), sodium lauryl sulfate, sorbitan monopalmitate and Triton X-100.

In a specific embodiment, at least one of the excipients used in an immunogenic composition of the invention is lutrol (e.g., Lutrol F 127). Preferably, the concentration of lutrol used in the immunogenic compositions of the invention may be from about 1% w/v to about 25% w/v, from about 3% w/v to about 15% w/v, or from about 5% w/v to about 10% w/v. Surfactants are typically used in the preparation and manufacturing of immunogenic compositions, particularly vaccines. In such cases, residual concentrations of the surfactant may be found in the final immunogenic composition, left over from the preparation or manufacturing of the composition. Such residual concentrations are too low to result in the adjuvant activity observed with the immunogenic compositions of the invention. Examples of such surfactants are octyl- or nonylphenoxy polyoxyethanols (e.g., Triton™ series), polyoxyethylene sorbitan esters (e.g., Tween™ series), and polyoxyethylene esters or ethers; Octylphenoxy polyoxyethanols and polyoxyethylene sorbitan esters including t-octylphenoxypolyoxyehtnaol; and Polyoxyethylene sorbitan esters including poloxyethylene sorbitan monooleate; Triton X-45, Triton X-102, Triton X-114, Triton X-165, Triton X-205, Triton X-305, Triton N-57, Triton N-101, Triton N-128, Breij 35, Laureth-9, Steareth-9, Tween 80™. (For a list of surfactants see, e.g., Surfactant Systems, eds., Attwood and Florence, 1983, Chapman and Hall, which is incorporated herein by reference in its entirety).

In another embodiment, at least one of the excipients used in an immunogenic composition of the invention is a suspending agent. Although not intending to be bound by a particular mechanism of action, a suspending agent increases the viscosity of the composition by for example reducing the rate of sedimentation of particles dispersed throughout a vehicle in which they are not soluble. Examples of suspending agents that may be used in the compositions of the invention include, but are not limited to, agar, bentonite, carbomer (e.g., Carbopol), carboxymethylcellulose sodium, gelatin, hydroxyethyl cellulose, hydroxypropyl cellulose, hydroxypropyl methylcellulose, kaolin, methylcellulose, tragacanth and veegum.

In a specific embodiment, at least one of the excipients used in the composition of the invention is methylcellulose. Preferably, the concentration of methylcellulose used in the immunogenic compositions of the invention may be from about 0.001% w/v to about 1% w/v, from about 0.01% w/v to about 0.5% w/v, or from about 0.02% w/v to about 0.1% w/v.

In another embodment, at least one of the excipients used in an immunogenic composition of the invention is a tonicity agent. Tonicity agents are particularly desired in the immunogenic compositions of the invention as they provide a solution with osmotic characteristics similar to physiologic fluid, and are thus optimal for injectable compositions of the invention. Examples of a tonicity agent that may be used in the immunogenic compositions of the invention include, but are not limited to, dextrose, glucose and sodium chloride.

In another embodiment, at least one of the excipients used in an immunogenic composition of the invention is a vehicle. As used herein, vehicle is a carrying agent for a substance in a pharmaceutical composition. Vehicles are frequently used in formulating a variety of compositions for oral and parenteral administration. Vehicles for use in the methods and immunogenic compositions of the invention may be aqueous or oleaginous vehicles. Examples of a vehicle which may be used in the immunogenic compositions of the invention include, but are not limited to, corn oil, mineral oil, peanut oil, sesame oil, bacteriostatic sodium chloride injection and bacteriostatic water.

In another embodiment, at least one of the excipients used in an immunogenic composition of the invention is a growth medium ingredient. Growth medium ingredients are particularly useful when the composition is a vaccine. Examples of growth medium ingredients that may be used in the immunogenic compositions and methods of the invention include, but are not limited to, amino acids, bactopeptone, bovine albumin, bovine serum, egg protein, human serum albumin, mouse serum proteins, MRC-5 cellular protein, ovalbumin, vitamins and yeast proteins.

Other compounds or agents such as, but not limited to, serum protein (e.g., apo-transferrin, fetuin), aprotinin, glycolic acid (a skin exfoliate), mannose and urea, may be used for the combination of excipients. Any supplemental protein may possess an adjuvant activity when used in accordance with the methods of the present invention and delivered to a subject. Supplemental proteins are particularly useful as adjuvants for DNA immunogens.

In a specific embodiment, at least one of the excipients used in an immunogenic composition of the invention is urea. Preferably, the concentration of urea used in the immunogenic compositions of the invention may be from about 0.01% w/v to about 10% w/v, from about 0.1% w/v to about 5% w/v, or from about 0.2% w/v to about 1% w/v.

In one specific embodiment, the immunogenic composition of the invention comprises the combination of a surfactant and a humectant. A specific combination is lutrol and sorbitol. Preferably, the concentration of lutrol used in the immunogenic compositions of the invention may be from about 1% w/v to about 25% w/v, from about 3% w/v to about 15% w/v, or from about 5% w/v to about 10% w/v. Preferably, the concentration of sorbitol used in the immunogenic compositions of the invention may be from about 0.5% w/v to about 25% w/v, from about 3% w/v to about 15% w/v, or from about 5% w/v to about 10% w/v.

In another specific embodiment, the immunogenic composition of the invention comprises the combination of a surfactant and a suspending agent. A specific combination is lutrol and methylcellulose. Preferably, the concentration of lutrol used in the immunogenic compositions of the invention may be from about 1% w/v to about 25% w/v, from about 3% w/v to about 15% w/v, or from about 5% w/v to about 10% w/v. Preferably, the concentration of methylcellulose used in the immunogenic compositions of the invention may be from about 0.001% w/v to about 1% w/v, from about 0.01% w/v to about 0.5% w/v, or from about 0.02% w/v to about 0.1% w/v.

In another specific embodiment, the immunogenic composition of the invention comprises the combination of a surfactant, in particular, lutrol, and urea. Preferably, the concentration of lutrol used in the immunogenic compositions of the invention may be from about 1% w/v to about 25% w/v, from about 3% w/v to about 15% w/v, or from about 5% w/v to about 10% w/v. Preferably, the concentration of urea used in the immunogenic compositions of the invention may be from about 0.01% w/v to about 10% w/v, from about 0.1% w/v to about 5% w/v, or from about 0.2% w/v to about 1% w/v.

In another embodiment, at least one of the excipients used in the immunogenic composition of this invention is a geling agent, such as Pluronic or Poloxamer, including, but not limited to, Pluronic F-127, Pluronic F-68, and Pluronic F108.

In another embodiment, at least one of the excipients used in the immunogenic composition of this invention is a mucoadhesive or bioadhesive, such as, but not limited to, polycarbophils, polyacrylic acid, carbopols, carbopol EX55, capricol, carbomers, polysaccharides, hyaluronic acid, chitosans, lectins, cellulose, methylcellulose, carboxymethylcellulose, hydroxypropyl methyl cellulose, sodium alginate, gelatin, pectin, acacia, and povidone. In a specific embodiment, at least one of the excipients used in the composition of the invention is chitosan, methylcellulose, or gelatin.

The excipients used in the immunogenic compositions of the invention can exist in a liquid, gas or solid form. Two or more excipients are used in combination to achieve an additive or a synergistic effect. In one embodiment, the concentration of the excipient in the immunogenic compositions of the invention does not include the residual concentration of the excipient that may be present from the preparation or manufacturing of the composition prior to preparation of the immunogenic composition in accordance with the methods of the instant invention.

5.2.2 Antigenic or Immunogenic Agent

Antigenic or immunogenic agents that may be used in the immunogenic composition of this invention include antigens from an animal, a plant, a bacteria, a protozoan, a parasite, a virus or a combination thereof. The antigenic or immunogenic agent for use in the immunogenic composition of this invention may be any substance that under appropriate conditions results in an immune response in a subject, including, but not limited to, polypeptides, peptides, proteins, glycoproteins, lipids, nucleic acids and polysaccharides.

The immunogenic composition of this invention may comprise one or more antigenic or immunogenic agents. The amount of the antigenic or immunogenic agent used in the compositions of this invention may vary depending on the chemical nature and the potency of the antigenic or immunogenic agent. Typically, the starting concentration of the antigenic or immunogenic agent in the composition of this invention is the amount that is conventionally used for eliciting the desired immune response, using the conventional routes of administration, e.g., intramuscular injection. The concentration of the antigenic or immunogenic agent in the composition of this invention is then adjusted, e.g., by dilution using a diluent, so that an effective protective immune response is achieved as assessed using standard methods known in the art and described herein.

The antigenic or immunogenic agent may be any viral peptide, protein, polypeptide, or a fragment thereof derived from a virus including, but not limited to, RSV-viral proteins, e.g., RSV F glycoprotein, RSV G glycoprotein, influenza viral proteins, e.g., influenza virus neuramimidase, influenza virus hemagglutinin, herpes simplex viral protein, e.g., herpes simplex virus glycoprotein including for example, gB, gC, gD, and gE.

The antigenic or immunogenic agent for use in the immunogenic composition of this invention may be an antigen of a pathogenic virus, including as examples and not by limitation: adenovirdiae (e.g., mastadenovirus and aviadenovirus), herpesviridae (e.g., herpes simplex virus 1, herpes simplex virus 2, herpes simplex virus 5, and herpes simplex virus 6), leviviridae (e.g., levivirus, enterobacteria phase MS2, allolevirus), poxyiridae (e.g., chordopoxyirinae, parapoxvirus, avipoxvirus, capripoxvirus, leporipoxvirus, suipoxvirus, molluscipoxvirus, and entomopoxyirinae), papovaviridae (e.g., polyomavirus and papillomavirus), paramyxoviridae (e.g., paramyxovirus, parainfluenza virus 1, mobillivirus (e.g., measles virus), rubulavirus (e.g., mumps virus), pneumonovirinae (e.g., pneumovirus, human respiratory syncytial virus), and metapneumovirus (e.g., avian pneumovirus and human metapneumovirus), picornaviridae (e.g., enterovirus, rhinovirus, hepatovirus (e.g., human hepatitis A virus), cardiovirus, and apthovirus, reoviridae (e.g., orthoreovirus, orbivirus, rotavirus, cypovirus, fijivirus, phytoreovirus, and oryzavirus), retroviridae (e.g., mammalian type B retroviruses, mammalian type C retroviruses, avian type C retroviruses, type D retrovirus group, BLV-HTLV retroviruses, lentivirus (e.g. human immunodeficiency virus 1 and human immunodeficiency virus 2), spumavirus), flaviviridae (e.g., hepatitis C virus), hepadnaviridae (e.g., hepatitis B virus), togaviridae (e.g., alphavirus, e.g., sindbis virus) and rubivirus (e.g., rubella virus), rhabdoviridae (e.g., vesiculovirus, lyssavirus, ephemerovirus, cytorhabdovirus, and necleorhabdovirus), arenaviridae (e.g., arenavirus, lymphocytic choriomeningitis virus, Ippy virus, and lassa virus), and coronaviridae (e.g., coronavirus and torovirus).

The antigenic or immunogenic agent used in the immunogenic composition of this invention may be an infectious disease agent including, but not limited to, influenza virus hemagglutinin (Genbank Accession No. J02132; Air, 1981, Proc. Natl. Acad. Sci. USA 78: 7639-7643; Newton et al., 1983, Virology 128: 495-501), human respiratory syncytial virus G glycoprotein (Genbank Accession No. Z33429; Garcia et al., 1994, J. Virol.; Collins et al., 1984, Proc. Natl. Acad. Sci. USA 81: 7683), core protein, matrix protein or any other protein of Dengue virus (Genbank Accession No. M19197; Hahn et al., 1988, Virology 162: 167-180), measles virus hemagglutinin (Genbank Accession No. M81899; Rota et al., 1992, Virology 188: 135-142), herpes simplex virus type 2 glycoprotein gB (Genbank Accession No. M14923; Bzik et al., 1986, Virology 155:322-333), poliovirus I VP1 (Emini et al., 1983, Nature 304:699), envelope glycoproteins of HIV I (Putney et al., 1986, Science 234: 1392-1395), hepatitis B surface antigen (Itoh et al., 1986, Nature 308: 19; Neurath et al., 1986, Vaccine 4: 34), diptheria toxin (Audibert et al., 1981, Nature 289: 543), streptococcus 24M epitope (Beachey, 1985, Adv. Exp. Med. Biol. 185:193), gonococcal pilin (Rothbard and Schoolnik, 1985, Adv. Exp. Med. Biol. 185:247), pseudorabies virus g50 (gpD), pseudorabies virus II (gpB), pseudorabies virus gIII (gpC), pseudorabies virus glycoprotein H, pseudorabies virus glycoprotein E, transmissible gastroenteritis glycoprotein 195, transmissible gastroenteritis matrix protein, swine rotavirus glycoprotein 38, swine parvovirus capsid protein, Serpulina hydodysenteriae protective antigen, bovine viral diarrhea glycoprotein 55, Newcastle disease virus hemagglutinin-neuramimidase, swine flu hemagglutinin, swine flu neuramimidase, foot and mouth disease virus, hog cholera virus, swine influenza virus, African swine fever virus, Mycoplasma hyopneumoniae, infectious bovine rhinotracheitis virus (e.g., infectious bovine rhinotracheitis virus glycoprotein E or glycoprotein G), or infectious laryngotracheitis virus (e.g., infectious laryngotracheitis virus glycoprotein G or glycoprotein I), a glycoprotein of La Crosse virus (Gonzales-Scarano et al., 1982, Virology 120: 42), neonatal calf diarrhea virus (Matsuno and Inouye, 1983, Infection and Immunity 39: 155), Venezuelan equine encephalomyelitis virus (Mathews and Roehrig, 1982, J. Immunol. 129: 2763), punta toro virus (Dalrymple et al., 1981, in Replication of Negative Strand Viruses, Bishop and Compans (eds.), Elsevier, N.Y., p. 167), murine leukemia virus (Steeves et al., 1974, J. Virol. 14:187), mouse mammary tumor virus (Massey and Schochetman, 1981, Virology 115: 20), hepatitis B virus core protein and/or hepatitis B virus surface antigen or a fragment or derivative thereof (see, e.g., U.K. Patent Publication No. GB 2034323A published Jun. 4, 1980; Ganem and Varmus, 1987, Ann. Rev. Biochem. 56:651-693; Tiollais et al., 1985, Nature 317:489-495), antigen of equine influenza virus or equine herpesvirus (e.g., equine influenza virus type A/Alaska 91 neuramimidase, equine influenza virus type A/Miami 63 neuramimidase, equine influenza virus type A/Kentucky 81 neuramimidase equine herpesvirus type 1 glycoprotein B, and equine herpesvirus type 1 glycoprotein D, antigen of bovine respiratory syncytial virus or bovine parainfluenza virus (e.g., bovine respiratory syncytial virus attachment protein (BRSV G), bovine respiratory syncytial virus fusion protein (BRSV F), bovine respiratory syncytial virus nucleocapsid protein (BRSV N), bovine parainfluenza virus type 3 fusion protein, and the bovine parainfluenza virus type 3 hemagglutinin neuramimidase), bovine viral diarrhea virus glycoprotein 48 or glycoprotein 53.

The antigenic or immunogenic agent in the immunogenic composition of this invention may also be a cancer antigen or a tumor antigen. Any cancer or tumor antigen known to one skilled in the art may be used in accordance with the immunogenic compositions of the invention including, but not limited to, KS ¼ pan-carcinoma antigen (Perez and Walker, 1990, J. Immunol. 142:3662-3667; Bumal, 1988, Hybridoma 7(4):407-415), ovarian carcinoma antigen (CA125) (Yu et al., 1991, Cancer Res. 51(2):468-475), prostatic acid phosphate (Tailor et al., 1990, Nucl. Acids Res. 18(16):4928), prostate specific antigen (Henttu and Vihko, 1989, Biochem. Biophys. Res. Comm. 160(2): 903-910; Israeli et al., 1993, Cancer Res. 53:227-230), melanoma-associated antigen p97 (Estin et al., 1989, J. Natl. Cancer Instit. 81(6):445-446), melanoma antigen gp75 (Vijayasardahl et al., 1990, J. Exp. Med. 171(4):1375-1380), high molecular weight melanoma antigen (HMW-MAA) (Natali et al., 1987, Cancer 59: 55-63; Mittelman et al., 1990, J. Clin. Invest. 86:2136-2144), prostate specific membrane antigen, carcinoembryonic antigen (CEA) (Foon et al., 1994, Proc. Am. Soc. Clin. Oncol. 13:294), polymorphic epithelial mucin antigen, human milk fat globule antigen, colorectal tumor-associated antigens such as: CEA, TAG-72 (Yokata et al., 1992, Cancer Res. 52:3402-3408), CO17-1A (Ragnhammar et al., 1993, Int. J. Cancer 53:751-758); GICA 19-9 (Herlyn et al., 1982, J. Clin. Immunol. 2:135), CTA-1 and LEA, Burkitt's lymphoma antigen-38.13, CD19 (Ghetie et al., 1994, Blood 83:1329-1336), human B-lymphoma antigen-CD20 (Reff et al., 1994, Blood 83:435-445), CD33 (Sgouros et al., 1993, J. Nucl. Med. 34:422-430), melanoma specific antigens such as ganglioside GD2 (Saleh et al., 1993, J. Immunol., 151, 3390-3398), ganglioside GD3 (Shitara et al., 1993, Cancer Immunol. Immunother. 36:373-380), ganglioside GM2 (Livingston et al., 1994, J. Clin. Oncol. 12:1036-1044), ganglioside GM3 (Hoon et al., 1993, Cancer Res. 53:5244-5250), tumor-specific transplantation type of cell-surface antigen (TSTA) such as virally-induced tumor antigens including T-antigen DNA tumor viruses and Envelope antigens of RNA tumor viruses, oncofetal antigen-alpha-fetoprotein such as CEA of colon, bladder tumor oncofetal antigen (Hellstrom et al., 1985, Cancer. Res. 45:2210-2188), differentiation antigen such as human lung carcinoma antigen L6, L20 (Hellstrom et al., 1986, Cancer Res. 46:3917-3923), antigens of fibrosarcoma, human leukemia T cell antigen-Gp37 (Bhattacharya-Chatterjee et al., 1988, J. of Immunospecifically. 141:1398-1403), neoglycoprotein, sphingolipids, breast cancer antigen such as EGFR (Epidermal growth factor receptor), HER2 antigen (p185^{HER2}), polymorphic epithelial mucin (PEM) (Hilkens et al., 1992, Trends in Bio. Chem. Sci. 17:359), malignant human lymphocyte antigen-APO-1 (Bernhard et al., 1989, Science 245:301-304), differentiation antigen (Feizi, 1985, Nature 314:53-57) such as I antigen found in fetal erythrocytes, primary endoderm, I antigen found in adult erythrocytes, preimplantation embryos, I(Ma) found in gastric adenocarcinomas, M18, M39 found in breast epithelium, SSEA-1 found in myeloid cells, VEP8, VEP9, Myl, VIM-D5, D₁56-22 found in colorectal cancer, TRA-1-85 (blood group H), C14 found in colonic adenocarcinoma, F3 found in lung adenocarcinoma, AH6 found in gastric cancer, Y hapten, Le^{y} found in embryonal carcinoma cells, TL5 (blood group A), EGF receptor found in A431 cells, E₁ series (blood group B) found in pancreatic cancer, FC10.2 found in embryonal carcinoma cells, gastric adenocarcinoma antigen, CO-514 (blood group Le^{a}) found in Adenocarcinoma, NS-10 found in adenocarcinomas, CO-43 (blood group Le^{b}), G49 found in EGF receptor of A431 cells, MH2 (blood group ALe^{b}/Le^{y}) found in colonic adenocarcinoma, 19.9 found in colon cancer, gastric cancer mucins, T₅A₇ found in myeloid cells, R₂₄ found in melanoma, 4.2, G_{D3}, D1.1, OFA-1, G_{M2}, OFA-2, G_{D2}, and M1:22:25:8 found in embryonal carcinoma cells, and SSEA-3 and SSEA-4 found in 4 to 8-cell stage embryos. In one embodiment, the antigen is a T cell receptor derived peptide from a Cutaneous T cell Lymphoma (see, Edelson, 1998, The Cancer Journal 4:62).

The antigenic or immunogenic agent in the immunogenic composition of this invention may comprise a virus, against which an immune response is desired. In certain cases, the immunogenic composition of this invention comprise recombinant or chimeric viruses. In other cases, the immunogenic composition of this invention comprises a virus which is attenuated. Production of recombinant, chimeric and attenuated viruses may be performed using standard methods known to one skilled in the art. This invention also encompasses a live recombinant viral vaccine or an inactivated recombinant viral vaccine to be formulated in accordance with the invention. A live vaccine may be preferred because multiplication in the host leads to a prolonged stimulus of similar kind and magnitude to that occurring in natural infections, and therefore, confers substantial, long-lasting immunity. Production of such live recombinant virus vaccine formulations may be accomplished using conventional methods involving propagation of the virus in cell culture or in the allantois of the chick embryo followed by purification.

The recombinant virus may be non-pathogenic to the subject to which it is administered. In this regard, the use of genetically engineered viruses for vaccine purposes may require the presence of attenuation characteristics in these strains. The introduction of appropriate mutations (e.g., deletions) into the templates used for transfection may provide the novel viruses with attenuation characteristics. For example, specific missense mutations which are associated with temperature sensitivity or cold adaptation can be made into deletion mutations. These mutations should be more stable than the point mutations associated with cold or temperature sensitive mutants and reversion frequencies should be extremely low.

Alternatively, chimeric viruses with “suicide” characteristics may be constructed for use in the composition of this invention. Such viruses would go through only one or a few rounds of replication within the host. When used as a vaccine, the recombinant virus would go through limited replication cycle(s) and induce a sufficient level of immune response but it would not go further in the human host and cause disease.

Alternatively, inactivated (killed) virus may be formulated in accordance with the invention. Inactivated vaccine formulations may be prepared using conventional techniques to “kill” the chimeric viruses. Inactivated vaccines are “dead” in the sense that their infectivity has been destroyed. Ideally, the infectivity of the virus is destroyed without affecting its immunogenicity. In order to prepare inactivated vaccines, the chimeric virus may be grown in cell culture or in the allantois of the chick embryo, purified by zonal ultracentrifugation, inactivated by formaldehyde or β-propiolactone, and pooled.

Completely foreign epitopes, including antigens derived from other viral or non-viral pathogens can also be engineered into the virus for use in the composition of this invention. For example, antigens of non-related viruses such as HIV (gp160, gp120, gp41) parasite antigens (e.g., malaria), bacterial or fungal antigens or tumor antigens can be engineered into the attenuated strain. Methods for production and manufacturing of vaccines are known to one skilled in the art and encompassed within the instant invention. Typically such methods include inoculating embryonated eggs, harvesting the allantoic fluid, concentrating, purifying and separating the whole virus, using for example zonal centrifugation, ultracentrifugation, ultrafiltration, and chromatography in a variety of combinations. Such methods encompass use of various chemicals for example as splitting agents (e.g., non-ionic surfactants, bile acids and derivatives thereof, alkyglycosides and derivatives thereof, acyl sugars), stabilizers, solvents, etc. In such cases, residual concentrations of these chemicals may be found in the final immunogenic composition, left over from the manufacturing and preparation of the vaccine compositions, however, such residual concentrations are not sufficient to result in an adjuvant activity of the vaccine compositions when it is delivered to the a subject. It should be emphasized that the concentration of the excipients of the invention as specified herein is greater than the residual concentration of such chemicals that may be present during the preparation and manufacturing of a vaccine composition.

Virtually any heterologous gene sequence may be constructed into the chimeric viruses for use in the immunogenic composition of this invention. Preferably, heterologous gene sequences are moieties and peptides that act as biological response modifiers. Preferably, epitopes that induce a protective immune response to any of a variety of pathogens, or antigens that bind neutralizing antibodies may be expressed by or as part of the chimeric viruses. For example, heterologous gene sequences that can be constructed into the chimeric viruses include, but are not limited to, influenza and parainfluenza hemagglutinin neuramimidase and fusion glycoproteins such as the HN and F genes of human PIV3. In addition, heterologous gene sequences that can be engineered into the chimeric viruses include those that encode proteins with immuno-modulating activities. Examples of immuno-modulating proteins include, but are not limited to, cytokines, interferon type 1, gamma interferon, colony stimulating factors, interleukin-1, -2, -4, -5, -6, -12, and antagonists of these agents.

Other heterologous sequences may be derived from tumor antigens, and the resulting chimeric viruses be used to generate an immune response against the tumor cells leading to tumor regression in vivo. In accordance with the present invention, recombinant viruses may be engineered to express tumor-associated antigens (TAAs), including but not limited to, human tumor antigens recognized by T cells (Robbins and Kawakami, 1996, Curr. Opin. Immunol. 8:628-636, incorporated herein by reference in its entirety); melanocyte lineage proteins, including gp100, MART-1/MelanA, TRP-1 (gp75) and tyrosinase; tumor-specific widely shared antigens, such as MAGE-1, MAGE-3, BAGE, GAGE-1, GAGE-1, N-acetylglucosaminyltransferase-V and p15; tumor-specific mutated antigens, such as β-catenin, MUM-1 and CDK4; non-melanoma antigens for breast, ovarian, cervical and pancreatic carcinoma, HER-2/neu, human papillomavirus-E6, -E7, MUC-1.

The antigenic or immunogenic agent for use in the immunogenic composition of this invention may include one or more of the select agents and toxins as identified by the Center for Disease Control. In certain cases, the select agent for use in the immunogenic composition of this invention may comprise one or more antigens from Staphyloccocal enterotoxin B, Botulinum toxin, protective antigen for Anthrax, and Yersinia pestis. A non-limiting examples of select agents and toxins for use in the immunogenic composition of this invention are listed in Table I:

TABLE I
SELECT AGENTS

HHS NON-OVERLAP SELECT AGENTS AND TOXINS
Crimean-Congo haemorrhagic fever virus
Coccidioides posadasii
Ebola viruses
Cercopithecine herpesvirus 1 (Herpes B virus)
Lassa fever virus
Marburg virus
Monkeypox virus
Rickettsia prowazekii
Rickettsia rickettsii
South American haemorrhagic fever viruses
Junin
Machupo
Sabia
Flexal
Guanarito
Tick-borne encephalitis complex (flavi) viruses
Central European tick-borne encephalitis
Far Eastern tick-borne encephalitis
Russian spring and summer encephalitis
Kyasanur forest disease
Omsk hemorrhagic fever
Variola major virus (Smallpox virus)
Variola minor virus (Alastrim)
Yersinia pestis
Abrin
Conotoxins
Diacetoxyscirpenol
Ricin
Saxitoxin
Shiga-like ribosome inactivating proteins
Tetrodotoxin
HIGH CONSEQUENCE LIVESTOCK PATHOGENS AND
TOXINS/SELECT AGENTS (OVERLAP AGENTS)
Bacillus anthracis
Brucella abortus
Brucella melitensis
Brucella suis
Burkholderia mallei (formerly Pseuodomonas mallei)
Burkholderia pseudomallei (formerly Pseuodomonas pseudomallei)
Botulinum neurotoxin producing species of Clostridium
Coccidioides immitis
Coxiella burnetii
Eastern equine encephalitis virus
Hendra virus
Francisella tularensis
Nipah Virus
Rift Valley fever virus
Venezuelan equine encephalitis virus
Botulinum neurotoxin
Clostridium perfringens epsilon toxin
Shigatoxin
Staphylococcal enterotoxin
T-2 toxin
USDA HIGH CONSEQUENCE LIVESTOCK PATHOGENS AND
TOXINS (NON-OVERLAP AGENTS AND TOXINS
Akabane virus
African swine fever virus
African horse sickness virus
Avian influenza virus (highly pathogenic)
Blue tongue virus (Exotic)
Bovine spongiform encephalopathy agent
Camel pox virus
Classical swine fever virus
Cowdria ruminantium (Heartwater)
Foot and mouth disease virus
Goat pox virus
Lumpy skin disease virus
Japanese encephalitis virus
Malignant catarrhal fever virus (Exotic)
Menangle virus
Mycoplasma capricolumi M.F38/M. mycoides capri
Mycoplasm mycoides mycoides
Newcastle disease virus (VVND)
Peste Des Petits Ruminants virus
Rinderpest virus
Sheep pox virus
Swine vesicular disease virus
Vesicular stomatitis virus (Exotic)
LISTED PLANT PATHOGENS
Liberobacter africanus
Liberobacter asiaticus
Peronosclerospora phillippinensis
Phakopsora pachyrhizi
Plum Pox Potyvirus
Ralstonia solanacearum race 3, biovar 2
Schlerophthora rayssiae var zeae
Synchytrium endobioticum
Xanthomonas oryzae
Xylella fastidiosa (citrus variegated chlorosis strain)

5.2.3 Influenza Virus Antigens

Preferred vaccine delivery systems of the invention are influenza virus vaccines, which may comprise one or more influenza virus antigens. Preferably, the influenza virus antigens used in the immunogenic composition of the invention are surface antigens, including, but not limited to, haemagglutinin and neuramimidase antigens or a combination thereof. The influenza virus antigens may form part of a whole influenza vaccine formulations. Alternatively, the influenza virus antigens can be present as purified or substantially purified antigens. Techniques for isolating and purifying influenza virus antigens are known to one skilled in the art and are contemplated in the present invention. An example of a haemagglutinin/neuramimidase preparation suitable for use in the compositions of the present invention is the “Fluvirin” product manufactured and sold by Evans Medical Limited of Speke, Merseyside, United Kingdom, and see also S. Renfrey and A. Watts, 1994 Vaccine, 12(8): 747-752; which is incorporated herein by reference in its entirety.

The influenza vaccines useful in the immunogenic compositions of the present invention may be any commercially available influenza vaccine, preferably a trivalent subunit vaccine, e.g., FLUZONE™ attenuated flu vaccine (Aventis Pasteur, Inc. Swiftwater, Pa.). The influenza vaccine formulations of the invention have a therapeutic efficacy at a dose which is lower than the conventional dose used for intramuscular delivery of influenza vaccines. The influenza vaccine used in the immunogenic composition of the invention may be a non-live influenza antigenic preparation, preferably a split influenza or a subunit antigenic preparation, prepared using common methods known in the art. Most preferably, the influenza vaccine used in accordance with the invention is a trivalent vaccine.

The invention encompasses influenza vaccine formulations comprising a non-live influenza antigenic preparation, preferably a split influenza preparation or a subunit antigenic preparation prepared from a live virus. Most preferably the influenza antigenic preparation is a split influenza antigenic preparation.

The influenza vaccine formulation of the invention may contain influenza virus antigens from a single viral strain, or from a plurality of strains. For example, the influenza vaccine formulation may contain antigens taken from up to three or more viral strains. Purely by way of example, the influenza vaccine formulation may contain antigens from one or more strains of influenza A, together with antigens from one or more strains of influenza B. Examples of influenza strains are strains of influenza A/Texas/36/91, A/Nanchang/933/95 and B/Harbin/7/94).

In a most preferred embodiment, the influenza vaccine formulation of the invention comprises a commercially available influenza vaccine, FLUZONE™, which is an attenuated flu vaccine (Connaught Laboratories, Swiftwater, Pa.). FLUZONE is a trivalent subvirion vaccine comprising 15 ug/dose of each the HA. For example the commerical trivalent vaccine may contain influenza A/Texas/36/91 (NINI), A/Beijing/32/92 (H3N2) and B/Panama, 45/90 viruses. The virus strains may change each year.

Preferably, the influenza vaccine formulations of the invention have a lower quantity of haemagglutinin than conventional vaccines and are administered in a lower volume. In some embodiments, the quantity of haemagglutinin per strain of influenza is about 1-7.5 μg, more preferably approximately 3 μg or approximately 5 μg, which is about one fifth or one third, respectively, of the dose of haemagglutinin used in conventional vaccines for intramuscular administration.

The volume of a dose of an influenza vaccine formulation according to the invention is between 0.025 ml and 2.5 ml, more preferably approximately 0.1 ml or approximately 0.2 ml. In a specific embodiment, the invention encompasses a 50 μl dose volume of the influenza vaccine. A 0.1 ml dose is approximately one fifth of the volume of a conventional intramuscular flu vaccine dose. The volume of liquid that can be administered depends in part upon the site of the injection. For example, for an injection in the deltoid region, 0.1 ml is the maximum preferred volume whereas in the lumbar region a large volume e.g. about 0.2 ml can be given.

Standards are applied internationally to measure the efficacy of influenza vaccines. The European Union official criteria for an effective vaccine against influenza are set out in the table below. Theoretically, to meet the European Union requirements, and thus be approved for sale in the EU, an influenza vaccine has to meet one of the criteria in the table below, for all strains of influenza included in the vaccine. However in practice, at least two or more, probably all three of the criteria will need to be met for all strains, particularly for a new vaccine coming onto the market. Under some circumstances, two criteria may be sufficient. For example, it may be acceptable for two of the three criteria to be met by all strains while the third criterion is met by some but not all strains (e.g., two out of three strains). The requirements are different for adult populations (18-60 years) and elderly populations (>60 years).

TABLE II
EU STANDARDS FOR AN EFFECTIVE
INFLUENZA VACCINE

| | 18-60 years | >60 years |
|---|---|---|
| Seroconversion rate | >40% | >30% |
| Conversion factor | >2.5 | >2.0 |
| Protection rate | >70% | >60% |

Seroconversion rate is defined as the percentage of vaccines who have at least a 4-fold increase in serum haemagglutinin inhibition (HI) titres after vaccination, for each vaccine strain. Conversion factor is defined as the fold increase in serum HI geometric mean titres (C3MTs) after vaccination, for each vaccine strain. Protection rate is defined as the percentage of vaccines with a serum HI titre equal to or greater than 1:40 after vaccination (for each vaccine strain) and is normally accepted as indicating protection.

The influenza vaccine formulations of the invention meet some or all of the EU criteria for influenza vaccines as set out hereinabove, such that the vaccine is approvable kin Europe. Preferably, at least two out of the three EU criteria are met, for the or all strains of influenza represented in the vaccine. More preferably, at least two criteria are met for all strains and the third criterion is met by all strains or at least by all but one of the strains. More preferably, all strains present meet all three of the criteria. Preferably, the influenza vaccine formulations of the invention additionally meet some or all criteria of the Federal Drug Administration and/or USPHS reequirements for the current influenza vaccines.

5.2.4 Geling Agents

In some embodiments, a component which may be used in the immunogenic compositions of the invention is a geling agent that polymerizes or gels once administered to a subject's tissue. Such geling agents preferably create a semi-solid to solid matrix, which may be two or three dimensional that may allow interaction of the antigenic or immunogenic agent with the biological and immunological space of the target tissue, specifically with the immune cells residing therein. In some embodiments, the geling agents enhance the presentation and/or availability of the antigenic or immunogenic agent within the biological and immunological space of the target tissue. Geling agents suitable for the immunogenic compositions of the invention preferably break down and/or degrade within the body of the subject to which they are administered, and do not result in any toxic, deleterious, or undesired effects on the subject.

In some embodiments, the geling agent may not gel and merely thickens, i.e., the viscosity of the molecule is increased as assessed visually. Regardless of the physical state of the geling agent below the liquid-gel transition temperature, the viscosity of the geling agent may increase by at least 30%, at least 50%, at least 60%, at least 80%, at least 90%, or at least 99% at a temperature above the transition temperature, e.g., at a physiological temperature.

The geling agent used in the immunogenic compositions of the invention preferably undergoes a thermally induced physical transition from a liquid to a gel as the temperature of the composition is increased over a temperature range consisting of a first temperature and a second temperature. Preferably, the first temperature is in a range from 1° C. to 20° C., and the second temperature is in the range of 25° C. to 37° C.

The geling agent used in the immunogenic compositions of the invention preferably undergoes a thermally induced liquid-gel transition at a physiological temperature of the subject to which the compositions are administed. In a specific embodiment, when the subject is human, the geling agent used in the immunogenic compositions of the invention is selected and formulated such that the composition undergoes a thermally induced liquid-gel transition at a temperature below 40° C., preferably below 37° C. In some embodiments, the geling agent undergoes a thermally induced liquid-gel transition at a temperature from about 10° C. to about 37° C., preferably at a temperature from about 25° C. to 37° C. Preferably, the liquid-gel transition of the immunogenic composition of the invention is accompanied by an increase in the viscosity of the immunogenic composition.

In a specific embodiment, the geling agent used in the immunogenic compositions of the invention is a polymer. Any biocompatible, biodegradable polymer may be used that as formulated in the composition of the invention is capable of imparting the desired liquid-gel transition property to the immunogenic composition. Non-limiting examples of some polymers useful for preparing the immunogenic compositions of the invention include polyethers, preferably polyoxyalkylene block copolymers, more preferably polyoxyalkylene block copolymers including polyoxyethylene-polyoxypropylene block copolymers referred to herein as POE-POP block copolymers, such as Pluronic™ F68, Pluronic™ F127, Pluronic™ L121, and Pluronic™ L101, and Tetronic™ T1501; and poly (ether-ester) block copolymers. Some examples of the above-identified polymers are disclosed in U.S. Pat. Nos. 5,702,717 and 5,861,174; which are incorporated herein by reference in their entirety.

The invention encompasses an immunogenic composition comprising more than one of the above identified polymers and/or other polymers that provide the desired characteristics, e.g., enhanced protective immune response when delivered to a subject. In some embodiments, the immunogenic composition may further comprise other polymers and/or other additives, to the extent the inclusion of the additional components is not inconsistent with performance requirements of the composition of the invention. Furthermore, these polymers may be combined, e.g., mixed with other polymers or other additives, such as sugars, to vary the liquid-gel transition temperature, typically in aqueous solutions.

Polyoxyalkylene block copolymers (Pluronic copolymer) are particularly preferred to use as the polymer in accordance with the invention. A polyoxyalkylene block copolymer is a polymer including at least one block (i.e., a polymer segment) of a first polyoxyalkylene and at least one block of a second polyoxyalkylene, although other blocks may be present as well.

In a specific embodiment of the invention, the polyoxyalkylene block copolymer comprises at least one block of a first polyoxyalkylene and at least one block of a second polyoxyalkylene. In yet another specific embodiment, the first polyoxylakylene is polyoxyethylene and the second polyoxyalkylene is polyoxypropylene.

POE-POP block copolymers are one class of preferred polyoxyalkylene block copolymers for use as the biocompatible polymer in the immunogenic compositions of the invention. These polymers can be designed and synthesized using variable amounts of the POE-POP blocks and with differential arrangement of the POP and POE blocks. Any of the polyoxyalkylene block copolymers known in the art are encompassed within the methods and formulations of the instant invention. For a review of polyoxyalkylene block copolymers, their molecular structure, synthesis, and purification see, e.g., Newman et al., 1998, Advanced Drug Delivery Reviews 32: 199-223; Verheul & Snippe, 1992, Res. Immunol. 143(5): 512-9; Hunter et al., 1994 AIDS Res. and Human Retroviruses, 10: Suppl. 2, S95-8; Newman et al., 1998, Crit. Rev. Ther. Drug. Carrier Syst. 15(2): 89-142; Kabanov et al., 2002 Advanced Drug Delivery Review 54: 223-233; Moghimi et al., 2000 TIBTECH, 18: 412-20; all of which are incorporated herein by reference in their entirety.

The polyoxyalkylene copolymers that may be used as a geling agent in the immunogenic compositions of the invention may be triblocks, e.g., L81, L92, L101, L121, L122, L141, L180, L185, reversed triblocks, e.g., 25R1, 31R1, octablocks, e.g., T1101, T1301, T1501, reversed octablocks, e.g., T130R1, T130R2, T150R1. The invention encompasses polyoxyalkylene copolymers wherein the orientation and size of the POP and POE blocks may be varied using common methods known in the art to achieve a desired surfactant property, depending on the composition being prepared. In a specific embodiment, the polyoxyalkylene copolymer used in the immunogenic composition of the invention is a linear molecule with the polymer blocks organized as POE-POP-POE.

The invention encompasses low molecular weight polyoxyalkylene copolymers as well as high molecular weight polyoxyalkylene copolymers. The low molecular weight copolymers may be about 2 to 6 KDa. The high molecular weight copolymers may be about 12 to 15 KDa. The copolymers used within the compositions of the invention may have adjuvant activity, e.g., enhance the therapeutic efficacy of a vaccine formulation. In a preferred embodiment, the polyoxyalkylene copolymers used in the immunogenic compositions of the invention are about 12 to 15 KDa. In yet another preferred embodiment, the polyoxyalkylene copolymers used in the immunogenic composition of the invention has a low POE concentration, preferably 10%, more preferably 8%, most preferably 5% so that optimal adjuvant activity is achieved. In a most preferred embodiment, the POE concentration of the polyoxyalkylene is no more than 5%.

The invention encompasses any of the pluronic copolymers that are commercially available, e.g., TiterMax® (CytRx Corporation, Atlanta, Ga.); Syntex Adjuvant formulation (Syntex Res., Palo Alto, Calif.). In preferred embodiments, the invention encompasses pluronic copolymers manufactured by Wyandotte Chemical Corporation and BASF Performance Chemicals (Parsiponny, N.J.), including, but not limited to, L31, L81, L92, L101, L121, L122, P102, F108, L141, L180, L185, P1004, and P1005.

In some embodiments, the invention encompasses the use of high molecular weight CRL copolymers, such as those commercially available from CytRx Corporation (Norcross, Ga.). The CRL copolymers are similar to pluronic copolymers in orientation of the POE and POP blcoks, however, they are significantly larger in size. CRL copolymers containin 9000-20,000 dalton POP cores flanked by POE blocks that constitue 2.5-20% of the total molecular weight. Any of the CRL copolymers known in the art are encompassed in the methods and compositions of the invention.

The concentration of the polymer used in the immunogenic compositions of the invention may be at least 0.1% (w/v), at least 1% (w/v), at least 10% (w/v), at least 15% (w/v), at least 20% (w/v), at least 25% (w/v), or at least 30% (w/v). In some embodiments, the concentration of the polymer used in the immunogenic compositions of the invention is less than 10% (w/v). In other embodiments, the concentration of the polymer used in the immunogenic compositions of the invention is more than 30% (w/v). The concentration of the polymer used in the immunogenic compositions of the invention is preferably the concentration at which an aqueous solution of the polymer gels, i.e., forms a semi-solid to solid two or three dimensional matrix at a physiological temperature, e.g., at 37° C. In some embodiments, the polymer used in the immunogenic compositions of the invention gels within 20 minutes or less, preferably within 10 minutes or less, and most preferably within 5 minutes or less at a physiological temperature, e.g., at 37° C., as assessed by visual inspection. Preferably, the concentration at which an aqueous solution of the polymer gels is also the concentration at which the therapeutic efficacy of the immunogenic composition of the invention is enhanced as determined using standard methods known in the art, e.g., as determined by the antibody response to the antigenic or immunogenic agent, relative to a control formulation, e.g., a formulation comprising the antigenic or immunogenic agent alone.

An exemplary method for determining the concentration of the polymer for the immunogenic compositions of the invention may comprise the following: an aqueous stock solution of the polymer is prepared; the solution is then incubated, preferably, by mechanical agitation, e.g., magnetic stirring, at a temperature below the liquid-gel transition temperature, e.g., on ice at 4° C.; the pH of the solution is adjusted to a physiological pH, ranging from 7.0 to 7.4, preferably to 7.2; the solution is then sterilized, preferably by filtration, e.g., using a 0.2 micron Gelman Acrodisc PF Syringe Filter # 4187; the solution is then incubated at 37° C., e.g., by placing it in a 37° C. water bath; and the solution is visually monitored. Specifically, the viscosity of the solution is visually monitored. In some embodiments, the solution gels within 5 minutes or less. In other embodiments, the solution gels within 20 minutes or less, 15 minutes or less, 10 minutes or less. If the solution does not gel within the time frame specified above, the concentration of the polymer may be adjusted so that a higher percentage of the polymer is used. The concentration of the polymer may be adjusted so that the solution preferably gels, as determined by visual inspection of the solution at a physiological temperature, e.g., 37° C.

In a specific embodiment, the invention encompasses the Lutrol F grade chemicals supplied by BASF Corporations including, but not limited to, F127, F68, F87, and F108. Preferably, the Lutrol F grade chemicals polymerize to form a gel at a physiological temperature, e.g., temperature ranging from 25° C. to 37° C., at a concentration ranging from about 10% (w/v) to 20% (w/v), from about 10% (w/v) to 25% (w/v), from about 10% (w/v) to about 30% (w/v), or from about 10% (w/v) to about 35% (w/v). Although not intending to be bound by a particular mechanism of action, polymerization of the Lutrol chemicals results in cross-linking, either covalently or non-covalently, of the chemical to form a two or three dimensional gelatinous matrix. The degree of polymerization may range from 5% to 50%, preferably 60% to 80%, most preferably about 90%.

In a specific embodiment, the Lutrol F grade used in the immunogenic compositions of the invention is F127, which forms a gelatinous matrix at a temperature of 37° C. and at a concentration of 20% (w/v). The polymerization of the F127 pluronic may be chemically and/or thermally induced. Preferably, the polymerization of the F127 pluronic is thermally induced.

In another specific embodiment, the Lutrol F grade used in the immunogenic compositions of the invention is F68, which forms a gelatinous matrix at a temperature of 37° C. and at a concentration of more than 30% (w/v). In yet another specific embodiment, the Lutrol F grade used in the immunogenic compositions of the invention is F108, which forms a gelatinous matrix at a temperature of 37° C., and at a concentration of 20% (w/v).

In some embodiments, the geling agent used in the immunogenic compositions of the invention polymerizes, e.g., forms a gel, at body temperature, i.e., a temperature ranging from 25°-37° C. Polymerization of the geling agent may be chemically and/or thermally induced. Although not intending to be bound by a particular mode of action, polymerization of the geling agent involves cross-linking, either covalently or non-covalently, of the polymer to form a two or three dimensional gelatinous matrix. The degree of polymerization may range from 5% to 50%, preferably 60% to 80%, most preferably about 90%. The geling agent used in accordance with the invention may be solid, liquid or a paste prior to the thermal and/or chemical change.

In other embodiments, the geling agent used in the immunogenic compositions of the invention has one or more biological properties of an adjuvant, when used in combination with another traditional excipient. As used herein, the term “adjuvant” refers to an auxiliary compound that, when present in an immunogenic composition, assists the active molecule, e.g., an immunogenic or antigenic agent in the composition, in producing the desired physiological response, e.g., enhancing the immune response to an antigenic or immunogenic agent. In yet other embodiments, the geling agent used in the immunogenic compositions of the invention has muco or bioadesive properties.

The amount of the geling agent that may be used in the immunogenic composition of the invention is typically from about 1% to 50% (w/v) of the composition, from about 15% (w/v) to about 30% (w/v), preferably from about 10% (w/v) to about 30% (w/v).

5.2.5 Muco or Bioadhesives

In certain embodiments, the molecule used in the immunogenic compositions of the invention is a muco or bioadhesive molecule, which may facilitate adherence of the antigenic or immunogenic agent to the biological and immunological surface of the target tissue, i.e., the surface of the immune cells. As used herein, bioadhesive or mucoadhesive means having the ability to adhere to a biological surface for an extended period of time. Preferably, such mucoadhesion or bioadhesion results in an enhancement of biological activity of the immunogenic compositions, e.g., enhanced therapeutic efficacy. Although not intending to be bound by a particular mechanism of action, muco or bioadhesion allows prolonged exposure of the immunogenic or antigenic agent in the compositions of the invention to the cells of the immune system, e.g., antigen presenting cells, residing in the target tissue. The adhesion property offered by the muco or bioadhesive molecule most likely leads to a prolonged residence time of the antigenic or immunogenic agent in the target tissue. Delivery of the antigenic or immunogenic agent benefits from mucoadhesion or bioadhesion by allowing adherence or “sticking” of the antigenic or immunogenic agent to the targeted biological surface. Furthermore, the antigenic or immunogenic agent may be held at the targeted biological surface thus allowing slow release of the antigenic or immunogenic agent, i.e., a depot effect.

Muco or bioadhesive molecules that may be used in the immunogenic compositions of the invention include, but are not limited to, polymers, e.g., polycarbophils polyacrylic acid (PAA), carobopols, capricol, Carbopol EX55, carbomers, polysaccharides, hyaluronic acid, chitosans; lectins; cellulose, methylcellulose, carboxymethylcellulose, hydroxypropyl methyl cellulose, sodium alginate, gelatin, pectin, acacia, povidone. For a review of available mucoadesive and bioadhesive molecules see reviews by Robinson et al., Annals New York Academy of Sciences, 307-314; Haas et al., 2002, Expert Opin. Biol. Ther. 2(3): 287-298; Woodley, 2001, Clin. Pharmacokin. 40(2): 77-84; Peppas et al., 1996, Biomaterials 17; 1553-61; all of which are incorporated herein by reference in their entirety.

The concentration of the bioadhesive or mucoadhesive molecule in the immunogenic compositions of the invention may be 0.1% (w/v) to 1% (w/v), 0.1% (w/v) to 5% (w/v), or 0.1% (w/v) to 10% (w/v), or 0.01% (w/v) to 10% (w/v), or 0.01% (w/v) to 0.04% (w/v). The concentration of the muco or bioadhesive molecule used in the immunogenic compositions of the invention is preferably the concentration at which the therapeutic efficacy of the composition is enhanced, e.g., as determined by the antibody response to the antigenic or immunogenic agent, relative to a control formulation, e.g., a formulation comprising the antigenic or immunogenic agent alone.

5.3 Preparation of the Vaccine Formulations

The immunogenic composition of this invention may be prepared by any method that results in a stable, sterile, injectable formulation. Preferably, the method for preparing an immunogenic composition of this invention comprises: providing solution(s) of the excipients; providing a solution of the antigenic or immunogenic agent; and combining the solutions of the excipients and the solution of the antigenic or immunogenic agent to form the inoculum, e.g., the solution to be injected to a subject. Two or more of the excipients may be prepared in one solution, or each excipient may be prepared in separate solutions.

In one embodiment, the excipients, in particulate forms, may be dissolved in a solution of the antigenic or immunogenic agent, such that a stable, sterile, injectable formulation is formed. Alternatively, the antigenic or immunogenic agent may be particulate and dissolved in the excipient solution such that a stable, sterile, injectable formulation is formed. For enhanced performance of the immunogenic composition of this invention, the antigenic or immunogenic agent should be uniformly dispersed throughout the composition.

In one embodiment, the excipients and the antigenic or immunogenic agent are mixed prior to administration to a subject. Alternatively, the excipients and the antigenic or immunogenic agent can be mixed during administration in a delivery device.

The amount of the antigenic or immunogenic agent used in the immunogenic composition of this invention may vary depending on the chemical nature and the potency of the antigenic or immunogenic agent and the specific excipients used. Typically, the starting concentration of the antigenic or immunogenic agent in the composition of this invention is the amount that is conventionally used for eliciting the desired immune response, using the conventional routes of administration, e.g., intramuscular injection. The concentration of the antigenic or immunogenic agent is then adjusted, e.g., by dilution using a diluent, in the immunogenic composition of the invention so that an effective protective immune response is achieved as assessed using standard methods known in the art and described herein.

The amount of the excipients used in the immunogenic composition of this invention may vary depending on the chemical nature of the excipients and the specific antigenic or immunogenic agent used. Certain preferred concentrations of the excipients, described in Section 5.2.1, above, can generally be used effectively with many antigenic or immunogenic agent. One of ordinary skill in the art would appreciate, however, that depending on the individual excipients and the antigenic or immunogenic agent, the amount of excipients may be adjusted using the methods that are substantially identical to those disclosed above for the determination of an effective amount of the antigenic or immunogenic agent, as well as other methods conventionally known in the art.

The immunogenic compositions of the present invention can be prepared as unit dosage forms. A unit dosage per vial may contain 0.1 mL to 1 mL, preferably 0.1 to 0.5 mL of the formulation. In some embodiments, a unit dosage form of the immunogenic compositions of the invention may contain 50 μL to 100 μL, 150 μL to 200 μL, or 250 μL to 500 μL of the formulation. If necessary, these preparations can be adjusted to a desired concentration by adding a sterile diluent to each vial. The immunogenic compositions of the invention are more effective in eliciting the desired immune response, and thus the total volume for delivery may be less than the volume that is conventionally used.

In some embodiments, the components of the immunogenic compositions of the invention, e.g., the antigenic or immunogenic agent and the excipients, are supplied either separately or mixed together in unit dosage form, for example, as a dry lyophilized powder or water free concentrate in a hermetically sealed container such as an ampoule or a sachette indicating the quantity of the active agent, e.g., the antigenic or immunogenic agent. In other embodiments, an ampoule of sterile diluent can be provided so that the components may be mixed prior to administration. In a specific embodiment, the excipients may be mixed with the antigenic or immunogenic agent just prior to administration. In another specific embodiment, the excipients may be mixed with the antigenic or immunogenic agent in a delivery device during administration.

The invention also provides immunogenic compositions that are packaged in a hermetically sealed container such as an ampoule or a sachette indicating the quantity of the components. In one embodiment, the immunogenic composition is supplied as a liquid, in another embodiment, as a dry sterilized lyophilized powder or water free concentrate in a hermetically sealed container and can be reconstituted, e.g., with water or saline to the appropriate concentration for administration to a subject. In an alternative embodiment, the immunogenic composition is supplied in liquid form in a hermetically sealed container indicating the quantity and concentration of the components. The immunogenic composition of the invention may be prepared by any method that results in a stable, sterile, injectable formulation.

The immunogenic compositions of the invention can be administered using any route of administration. Examples include, but are not limited to, intradermal, epidermal, intramuscular, transdermal, subcutaneous, junctional, and nasal administrations. The immunogenic compositions of the invention can be effectively administered using any well-known conventional methods known in the art.

The immunogenic compositions of the invention have little or no short term and/or long term toxicity when administered in accordance with the invention. In some instances, the immunogenic compositions of the invention, when administered to a subject, may have an undesired reaction at the site of the injection, e.g., skin irritation, swelling, rash, necrosis, skin sensitization. In these instances, one or more other excipients are used in the immunogenic compositions of the invention other than the excipients already used, which results in eliminating or reducing the undesired reaction at the site of injection. In other embodiments, the immunogenic compositions of the invention, when administered to a subject, have no undesired reaction at the site of the injection.

5.4 Determination of Efficacy of the Immunogenic Compositions

The invention encompasses methods for determining the efficacy of the immunogenic compositions using any standard method known in the art or described herein. Assays for determining the efficacy of the immunogenic compositions of the invention may be in vitro based assays or in vivo based assays, including animal based assays. In some embodiments, the invention encompasses detecting and/or quantitating a humoral immune response against the antigenic or immunogenic agent of a composition of the invention in a sample, e.g., serum or mucosal wash, obtained from a subject who has been administered an immunogenic composition of the invention. Preferably, the humoral immune response of the immunogenic compositions of the invention are compared to a control sample obtained from the same subject prior to administration with the inventive formulation or after an individual has been administered a control formulation, e.g., a formulation which simply comprises of the antigenic or immunogenic agent.

Assays for measuring humoral immune response are well known in the art; e.g., see, Coligan et al., (eds.), 1997, Current Protocols in Immunology, John Wiley and Sons, Inc., Section 2.1. A humoral immune response may be detected and/or quantitated using standard methods known in the art including, but not limited to, an ELISA assay. The humoral immune response may be measured by detecting and/or quantitating the relative amount of an antibody which specifically recognizes an antigenic or immunogenic agent in the sera of a subject who has been treated with an immunogenic composition of this invention relative to the amount of the antibody in an untreated subject. ELISA assays can be used to determine total antibody titers in a sample obtained from a subject treated with a composition of the invention. In other embodiments, ELISA assays may be used to determine the level of specific antibody isotypes and antibodies to neutralizing epitopes using methods known in the art.

ELISA based assays comprise preparing an antigen, coating the well of a 96 well microtiter plate with the antigen, adding test and control samples containing antigen specific antibody, adding a detector antibody specific to the antibody in test and control samples that is conjugated to an enzyme (e.g., horseradish peroxidase or alkaline phosphatase) and incubating for a period of time, and detecting the presence of the antigen with a color yielding substrate. One of skill in the art would be knowledgeable as to the parameters that can be modified to increase the signal detected as well as other variations of ELISAs known in the art. For further discussion regarding ELISAs see, e.g., Ausubel et al., eds, 1994, Current Protocols in Molecular Biology, Vol. 1, John Wiley & Sons, Inc., New York at 11.2.1.

In the cases where the immunogenic composition comprises an influenza antigen, any method known in the art for the detection and/or quantitation of an antibody response against an influenza antigen is encompassed within the methods of the invention. An exemplary method for determining an influenza antigen directed antibody response may comprise the following: an influenza antigen is used to coat a microtitre plate (Nunc plate); sera from a subject treated with an influenza vaccine formulation of the invention is added to the plate; antisera (containing 2^{nd} antibody) is added to the plate and incubated for a sufficient time to allow a complex to be formed, i.e., a complex between an antibody in the sera and the antisera. The complex is then detected using standard methods in the art. For exemplary assays for measuring an influenza specific antibody response, see, e.g., Newman et al., 1997, Mechanism of Aging & Development, 93: 189-203; Katz et al., 2000, Vaccine, 18: 2177-87; Todd et al., (Brown and Haaheim, eds.), 1998 in Modulation of the Immune Response to Vaccine Antigens, Dev. Biol. Stand. Basel, Karger, 92: 341-51; Kendal et al., 1982, in Concepts and Procedures for Laboratory-based Influenza Surveillance, Atlanta: CDC, B17-35; Rowe et al., 1999, J. Clin. Micro. 37: 937-43; Todd et al., 1997, Vaccine 15: 564-70; WHO Collaborating Centers for Reference and Research on Influenza, in Concepts and Procedures for Laboratory-based Influenza Surveillance, 1982, p. B-23; all of which are incorporated herein by reference in their entirety.

Furthermore, when the vaccine formulation comprises an influenza antigen, any method known in the art for the detection and/or quantitation levels of antibody with hemagglutination activity are encompassed within the invention. The hemagglutination inhibition assays are based on the ability of influenza viruses to agglutinate erythrocytes and the ability of specific HA antibodies to inhibit agglutination. Any of the hemagglutination inhibition assays known in the art are encompassed within the methods of the inventions, such as those disclosed in Newman et al., 1997, Mechanism of Aging & Development, 93: 189-203; Kendal et al., 1982, in Concepts and Procedures for Laboratory-based Influenza Surveillance, Atlanta: CDC, B 17-35; all of which are incorporated herein by reference in their entirety.

An exemplary hemagglutination inhibition assay comprises the following: sera from subjects treated with an influenza vaccine formulation of the invention are added to microtitre plates; HI-antigenic preparation containing 8 HA units is added to the plates; the mixture is mixed well by gently tapping the plates, and incubated for about 1 hour at 4° C.; erythrocyte suspension, e.g., 0.5% chicken erythrocytes, is added to the micotitre plate and the contents are mixed well by gently tapping the plates; the plates are further incubated at 4° C. until the cell control shows the button of normal settling; controls only contains PBS). Preferably, the serum samples are treated with inhibitors, such as neuramimidase or potassium periodate, to prevent non-specific inhibition of agglutination by serum factors. The HI titer is defined as the dilution factor of the highest dilution of serum that completely inhibits hemagglutination. This is determined by tilting the plates and observing the tear shaped streaming of cells that flow at the same rate as control cells.

The invention encompasses methods for determining the efficacy of the compositions of the invention by measuring cell-mediate immune response. Methods for measuring cell-mediated immune response are known to one skilled in the art and encompassed within the invention. In some embodiments, a T cell immune response may be measured for quantitating the immune response in a subject, for example by measuring cytokine production using common methods known to one skilled in the art including but not limited to ELISA from tissue culture supernatants, flow cytometry based intracellular cytokine staining of cells ex vivo or after an in vitro culture period, and cytokine bead array flow cytometry based assay. In yet other embodiments, the invention encompasses measuring T cell specific responses using common methods known in the art, including but not limited to chromium based release assay, flow cytometry based tetramer or dimer staining assay using known CTL epitopes.

5.5 Prophylactic and Therapeutic Uses

The invention provides methods of treatment and prophylaxis which involve administering an immunogenic composition of the invention to a subject, preferably a mammal, and most preferably a human for treating, managing or ameliorating symptoms associated with a disease or disorder, especially an infectious disease or cancer. The subject is preferably a mammal such as a non-primate, e.g., cow, pig, horse, cat, dog, rat, mouse and a primate, e.g., a monkey such as a Cynomolgous monkey and a human. In a preferred embodiment, the subject is a human. Preferably, the immunogenic composition of the invention is a vaccine composition.

The invention encompasses a method for immunization and/or stimulating an immune response in a subject comprising delivering a single dose of a composition of the invention to a subject, preferably a human. In some embodiments, the invention encompasses one or more booster immunizations. The immunogenic composition of the invention is particularly effective in stimulating and/or up-regulating an antibody response to a level greater than that seen in conventional immunogenic compositions (such as vaccines) and administration schedules. For example, an immunogenic composition of the invention may lead to an antibody response comprising generations of one or more antibody classes, such as IgM, IgG, and/or IgA. Most preferably, the immunogenic compositions of the invention including vaccine formulations stimulate a systemic immune response that protects the subject from at least one pathogen. The immunogenic compositions of the invention including vaccine compositions may provide systemic, local, or mucosal immunity or a combination thereof.

5.5.1 Target Diseases

The invention encompasses the treatment and/or prevention of an infectious disease in a subject, preferably a human, using an immunogenic composition of the invention. Infectious diseases that can be treated or prevented by the methods of the present invention are caused by infectious agents including, but not limited to, viruses, bacteria, fungi protozoa, helminths, and parasites.

Examples of viruses that have been found in humans and can be treated by the vaccine delivery systems of the invention include, but are not limited to, Retroviridae (e.g., human immunodeficiency viruses, such as HIV-1 (also referred to as HTLV-III, LAV or HTLV-III/LAV, or HIV-III; and other isolates, such as HIV-LP); Picornaviridae (e.g., polio viruses, hepatitis A virus; enteroviruses, human Coxsackie viruses, rhinoviruses, echoviruses); Calciviridae (e.g., strains that cause gastroenteritis); Togaviridae (e.g., equine encephalitis viruses, rubella viruses); Flaviridae (e.g., dengue viruses, encephalitis viruses, yellow fever viruses); Coronaviridae (e.g., coronaviruses); Rhabdoviridae (e.g., vesicular stomatitis viruses, rabies viruses); Filoviridae (e.g., ebola viruses); Paramyxoviridae (e.g., parainfluenza viruses, mumps virus, measles virus, respiratory syncytial virus); Orthomyxoviridae (e.g., influenza viruses); Bungaviridae (e.g., Hantaan viruses, bunga viruses, phleboviruses and Nairo viruses); Arena viridae (e.g., hemorrhagic fever viruses); Reoviridae (e.g., reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxyiridae (variola viruses, vaccinia viruses, pox viruses); and Iridoviridae (e.g. African swine fever virus); and unclassified viruses (e.g. the etiological agents of Spongiform encephalopathies, the agent of delta hepatitis (thought to be a defective satellite of hepatitis B virus), the agents of non-A, non-B hepatitis (class 1=internally transmitted; class 2=parenterally transmitted, e.g., Hepatitis C); Norwalk and related viruses, and astroviruses.

Retroviruses that results in infectious diseases in animals and humans and can be treated and/or prevented using the delivery systems and methods of the invention include both simple retroviruses and complex retroviruses. The simple retroviruses include the subgroups of B-type retroviruses, C-type retroviruses and D-type retroviruses. An example of a B-type retrovirus is mouse mammary tumor virus (MMTV). The C-type retroviruses include subgroups C-type group A (including Rous sarcoma virus (RSV), avian leukemia virus (ALV), and avian myeloblastosis virus (AMV)) and C-type group B (including murine leukemia virus (MLV), feline leukemia virus (FeLV), murine sarcoma virus (MSV), gibbon ape leukemia virus (GALV), spleen necrosis virus (SNV), reticuloendotheliosis virus (RV) and simian sarcoma virus (SSV)). The D-type retroviruses include Mason-Pfizer monkey virus (MPMV) and simian retrovirus type 1 (SRV-1). The complex retroviruses include the subgroups of lentiviruses, T-cell leukemia viruses and the foamy viruses. Lentiviruses include HIV-1, but also include HIV-2, SIV, Visna virus, feline immunodeficiency virus (FIV), and equine infectious anemia virus (EIAV). The T-cell leukemia viruses include HTLV-1, HTLV-II, simian T-cell leukemia virus (STLV), and bovine leukemia virus (BLV). The foamy viruses include human foamy virus (HFV), simian foamy virus (SFV) and bovine foamy virus (BFV).

Examples of RNA viruses that are antigens in vertebrate animals include, but are not limited to, the following: members of the family Reoviridae, including the genus Orthoreovirus (multiple serotypes of both mammalian and avian retroviruses), the genus Orbivirus (Bluetongue virus, Eugenangee virus, Kemerovo virus, African horse sickness virus, and Colorado Tick Fever virus), the genus Rotavirus (human rotavirus, Nebraska calf diarrhea virus, murine rotavirus, simian rotavirus, bovine or ovine rotavirus, avian rotavirus); the family Picornaviridae, including the genus Enterovirus (poliovirus, Coxsackie virus A and B, enteric cytopathic human orphan (ECHO) viruses, hepatitis A virus, Simian enteroviruses, Murine encephalomyelitis (ME) viruses, Poliovirus muris, Bovine enteroviruses, Porcine enteroviruses, the genus Cardiovirus (Encephalomyocarditis virus (EMC), Mengovirus), the genus Rhinovirus (Human rhinoviruses including at least 113 subtypes; other rhinoviruses), the genus Apthovirus (Foot and Mouth disease (FMDV); the family Calciviridae, including Vesicular exanthema of swine virus, San Miguel sea lion virus, Feline picornavirus and Norwalk virus; the family Togaviridae, including the genus Alphavirus (Eastern equine encephalitis virus, Semliki forest virus, Sindbis virus, Chikungunya virus, O'Nyong-Nyong virus, Ross river virus, Venezuelan equine encephalitis virus, Western equine encephalitis virus), the genus Flavirius (Mosquito borne yellow fever virus, Dengue virus, Japanese encephalitis virus, St. Louis encephalitis virus, Murray Valley encephalitis virus, West Nile virus, Kunjin virus, Central European tick borne virus, Far Eastern tick borne virus, Kyasanur forest virus, Louping III virus, Powassan virus, Omsk hemorrhagic fever virus), the genus Rubivirus (Rubella virus), the genus Pestivirus (Mucosal disease virus, Hog cholera virus, Border disease virus); the family Bunyaviridae, including the genus Bunyvirus (Bunyamwera and related viruses, California encephalitis group viruses), the genus Phlebovirus (Sandfly fever Sicilian virus, Rift Valley fever virus), the genus Nairovirus (Crimean-Congo hemorrhagic fever virus, Nairobi sheep disease virus), and the genus Uukuvirus (Uukuniemi and related viruses); the family Orthomyxoviridae, including the genus Influenza virus (Influenza virus type A, many human subtypes); Swine influenza virus, and Avian and Equine Influenza viruses; influenza type B (many human subtypes), and influenza type C (possible separate genus); the family paramyxoviridae, including the genus Paramyxovirus (Parainfluenza virus type 1, Sendai virus, Hemadsorption virus, Parainfluenza viruses types 2 to 5, Newcastle Disease Virus, Mumps virus), the genus Morbillivirus (Measles virus, subacute sclerosing panencephalitis virus, distemper virus, Rinderpest virus), the genus Pneumovirus (respiratory syncytial virus (RSV), Bovine respiratory syncytial virus and Pneumonia virus of mice); forest virus, Sindbis virus, Chikungunya virus, O'Nyong-Nyong virus, Ross river virus, Venezuelan equine encephalitis virus, Western equine encephalitis virus), the genus Flavirius (Mosquito borne yellow fever virus, Dengue virus, Japanese encephalitis virus, St. Louis encephalitis virus, Murray Valley encephalitis virus, West Nile virus, Kunjin virus, Central European tick borne virus, Far Eastern tick borne virus, Kyasanur forest virus, Louping III virus, Powassan virus, Omsk hemorrhagic fever virus), the genus Rubivirus (Rubella virus), the genus Pestivirus (Mucosal disease virus, Hog cholera virus, Border disease virus); the family Bunyaviridae, including the genus Bunyvirus (Bunyamwera and related viruses, California encephalitis group viruses), the genus Phlebovirus (Sandfly fever Sicilian virus, Rift Valley fever virus), the genus Nairovirus (Crimean-Congo hemorrhagic fever virus, Nairobi sheep disease virus), and the genus Uukuvirus (Uukuniemi and related viruses); the family Orthomyxoviridae, including the genus Influenza virus (Influenza virus type A, many human subtypes); Swine influenza virus, and Avian and Equine Influenza viruses; influenza type B (many human subtypes), and influenza type C (possible separate genus); the family paramyxoviridae, including the genus Paramyxovirus (Parainfluenza virus type 1, Sendai virus, Hemadsorption virus, Parainfluenza viruses types 2 to 5, Newcastle Disease Virus, Mumps virus), the genus Morbillivirus (Measles virus, subacute sclerosing panencephalitis virus, distemper virus, Rinderpest virus), the genus Pneumovirus (respiratory syncytial virus (RSV), Bovine respiratory syncytial virus and Pneumonia virus of mice); the family Rhabdoviridae, including the genus Vesiculovirus (VSV), Chandipura virus, Flanders-Hart Park virus), the genus Lyssavirus (Rabies virus), fish Rhabdoviruses, and two probable Rhabdoviruses (Marburg virus and Ebola virus); the family Arenaviridae, including Lymphocytic choriomeningitis virus (LCM), Tacaribe virus complex, and Lassa virus; the family Coronoaviridae, including Infectious Bronchitis Virus (IBV), Mouse Hepatitis virus, Human enteric corona virus, and Feline infectious peritonitis (Feline coronavirus).

Illustrative DNA viruses that are antigens in vertebrate animals include, but are not limited to: the family Poxyiridae, including the genus Orthopoxvirus (Variola major, Variola minor, Monkey pox Vaccinia, Cowpox, Buffalopox, Rabbitpox, Ectromelia), the genus Leporipoxvirus (Myxoma, Fibroma), the genus Avipoxvirus (Fowlpox, other avian poxvirus), the genus Capripoxvirus (sheeppox, goatpox), the genus Suipoxvirus (Swinepox), the genus Parapoxvirus (contagious postular dermatitis virus, pseudocowpox, bovine papular stomatitis virus); the family Iridoviridae (African swine fever virus, Frog viruses 2 and 3, Lymphocystis virus of fish); the family Herpesviridae, including the alpha-Herpesviruses (Herpes Simplex Types 1 and 2, Varicella-Zoster, Equine abortion virus, Equine herpes virus 2 and 3, pseudorabies virus, infectious bovine keratoconjunctivitis virus, infectious bovine rhinotracheitis virus, feline rhinotracheitis virus, infectious laryngotracheitis virus) the Beta-herpesviruses (Human cytomegalovirus and cytomegaloviruses of swine, monkeys and rodents); the gamma-herpesviruses (Epstein-Barr virus (EBV), Marek's disease virus, Herpes saimiri, Herpesvirus ateles, Herpesvirus sylvilagus, guinea pig herpes virus, Lucke tumor virus); the family Adenoviridae, including the genus Mastadenovirus (Human subgroups A,B,C,D,E and ungrouped; simian adenoviruses (at least 23 serotypes), infectious canine hepatitis, and adenoviruses of cattle, pigs, sheep, frogs and many other species, the genus Aviadenovirus (Avian adenoviruses); and non-cultivatable adenoviruses; the family Papoviridae, including the genus Papillomavirus (Human papilloma viruses, bovine papilloma viruses, Shope rabbit papilloma virus, and various pathogenic papilloma viruses of other species), the genus Polyomavirus (polyomavirus, Simian vacuolating agent (SV-40), Rabbit vacuolating agent (RKV), K virus, BK virus, JC virus, and other primate polyoma viruses such as Lymphotrophic papilloma virus); the family Parvoviridae including the genus Adeno-associated viruses, the genus Parvovirus (Feline panleukopenia virus, bovine parvovirus, canine parvovirus, Aleutian mink disease virus, etc). Finally, DNA viruses may include viruses which do not fit into the above families such as Kuru and Creutzfeldt-Jacob disease viruses and chronic infectious neuropathic agents.

Bacterial infections or diseases that can be treated or prevented by the methods of the present invention are caused by bacteria including, but not limited to, bacteria that have an intracellular stage in its life cycle, such as mycobacteria (e.g., Mycobacteria tuberculosis, M. bovis, M. avium, M. leprae, or M. africanum), rickettsia, mycoplasma, chlamydia, and legionella. Other examples of bacterial infections contemplated include but are not limited to infections caused by Gram positive bacillus (e.g., Listeria, Bacillus such as Bacillus anthracis, Erysipelothrix species), Gram negative bacillus (e.g., Bartonella, Brucella, Campylobacter, Enterobacter, Escherichia, Francisella, Hemophilus, Klebsiella, Morganella, Proteus, Providencia, Pseudomonas, Salmonella, Serratia, Shigella, Vibrio, and Yersinia species), spirochete bacteria (e.g., Borrelia species including Borrelia burgdorferi that causes Lyme disease), anaerobic bacteria (e.g., Actinomyces and Clostridium species), Gram positive and negative coccal bacteria, Enterococcus species, Streptococcus species, Pneumococcus species, Staphylococcus species, Neisseria species. Specific examples of infectious bacteria include but are not limited to: Helicobacter pyloris, Borelia burgdorferi, Legionella pneumophilia, Mycobacteria tuberculosis, M. avium, M. intracellulare, M. kansaii, M. gordonae, Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus viridans, Streptococcus faecalis, Streptococcus bovis, Streptococcus pneumoniae, Haemophilus influenzae, Bacillus antracis, corynebacterium diphtheriae, Erysipelothrix rhusiopathiae, Clostridium perfringers, Clostridium tetani, Enterobacter aerogenes, Klebsiella pneumoniae, Pasturella multocida, Fusobacterium nucleatum, Streptobacillus moniliformis, Treponema pallidium, Treponema pertenue, Leptospira, Rickettsia, and Actinomyces israelli.

Fungal diseases that can be treated or prevented by the methods of the present invention include but not limited to aspergilliosis, crytococcosis, sporotrichosis, coccidioidomycosis, paracoccidioidomycosis, histoplasmosis, blastomycosis, zygomycosis, and candidiasis.

Parasitic diseases that can be treated or prevented by the methods of the present invention including, but not limited to, amebiasis, malaria, leishmania, coccidia, giardiasis, cryptosporidiosis, toxoplasmosis, and trypanosomiasis. Also encompassed are infections by various worms, such as but not limited to ascariasis, ancylostomiasis, trichuriasis, strongyloidiasis, toxoccariasis, trichinosis, onchocerciasis. filaria, and dirofilariasis. Also encompassed are infections by various flukes, such as but not limited to schistosomiasis, paragonimiasis, and clonorchiasis. Parasites that cause these diseases can be classified based on whether they are intracellular or extracellular. An “intracellular parasite” as used herein is a parasite whose entire life cycle is intracellular. Examples of human intracellular parasites include Leishmania spp., Plasmodium spp., Trypanosoma cruzi, Toxoplasma gondii, Babesia spp., and Trichinella spiralis. An “extracellular parasite” as used herein is a parasite whose entire life cycle is extracellular. Extracellular parasites capable of infecting humans include Entamoeba histolytica, Giardia lamblia, Enterocytozoon bieneusi, Naegleria and Acanthamoeba as well as most helminths. Yet another class of parasites is defined as being mainly extracellular but with an obligate intracellular existence at a critical stage in their life cycles. Such parasites are referred to herein as “obligate intracellular parasites”. These parasites may exist most of their lives or only a small portion of their lives in an extracellular environment, but they all have at least one obligate intracellular stage in their life cycles. This latter category of parasites includes Trypanosoma rhodesiense and Trypanosoma gambiense, Isospora spp., Cryptosporidium spp, Eimeria spp., Neospora spp., Sarcocystis spp., and Schistosoma spp.

The invention also encompasses vaccine compositions to treat and/or prevent cancers, including, but not limited to, neoplasms, tumors, metastases, or any disease or disorder characterized by uncontrolled cell growth. For example, but not by way of limitation, cancers and tumors associated with the cancer and tumor antigens listed supra in Section 5.2.2 may be treated and/or prevented using the vaccine compositions of the invention.

5.6 Kits

The invention further comprises kits comprising an immunogenic composition of the invention as described herein. Optionally, kits of the invention may contain one or more delivery device appropriate for the route of delivery contemplated by the specific immunogenic composition contained therewith. In some embodiments, the invention also provides a pharmaceutical pack or kit comprising an immunogenic composition of the invention. In a specific embodiment, the invention provides a kit comprising, one or more containers filled with one or more of the components of the immunogenic compositions of the invention, e.g., an antigenic or immunogenic agent, excipients, and other optional components. In another specific embodiment, the kit comprises two containers, one containing an antigenic or immunogenic agent, and the other containing the combination of excipients. In some embodiment, a plurality of containers, each containing one or more of the excipients may be provided. Associated with such container(s) can be a notice in the form prescribed by a governmental agency regulating the manufacture, use or sale of pharmaceuticals or biological products, which notice reflects approval by the agency of manufacture, use or sale for human administration.

### 6. EXAMPLES

6.1 Preparation of Inoculum

Prior to preparation of various formulations, the pH of all excipient stock solutions were checked for a neutral pH, i.e., 7.0-7.4. The pH of the solutions was adjusted to neutral as necessary using dilute HCl or NaOH. All excipient stock solutions were sterile filtered through a 0.2 micron Gelman Acrodisc PF syringe filter #4187.

Aventis Fluzone® containing New Caledonia A Strain, Panama A strain, and Hong Kong B Strain, as commercially available, was used for inoculums. Test inoculums were prepared by adding appropriate amount of Aventis Fluzone® vaccine, and the excipients at a final concentration as denoted in the table below. Sodium chloride at 9% w/v was used to adjust the volume. A control inoculum was prepared by adding sodium chloride to the appropriate amount of respective Fluzone™ to yield the same final volume as the inoculums.

| Excipient Combination | Concentration | |
|---|---|---|
| Lutrol F127 and | 15% or 5% | w/v |
| Methylcellulose | 0.18% | w/v |
| Lutrol F127 and | 5% | w/v |
| Sorbitol | 5% | w/v |
| Lutrol F127 and | 5% | w/v |
| Urea | 0.2% | w/v |
| Gelatin and | 0.225% | w/v |
| Methylcellulose | 0.18% | w/v |
| Lutrol and | 5% | w/v |
| Gelatin | 0.225% | w/v |

6.2 Preparation of Chicken Red Blood Cells

Chicken Red Blood Cells (CRBC, 5 ml packed) were obtained from Charles River Laboratories (Cat. # S8776). cRBC was equally distribuited into four Flacon® Blue Max™ 50 ml polyethylene conical tubes, and centrifuged at 1500 rpm for 5-7 minutes at 4° C. Shipping buffer was removed from cRBC. Sodium chloride solution (0.9%) was added in 5 ml increments onto the cRBC pellet, and the pellet was resuspended. Combining the resuspended pellets from two of the first-wash, the volume was adjusted to 45 ml with sodium chloride solution (0.9%). The mixture was centrifuged at 1500 rpm for 5-7 minutes at 4° C., and the supernatant was discarded. Again, sodium chloride solution (0.9%) was added in 5 ml increments onto the cRBC pellet, and the pellet was resuspended. The resuspended pelletes from two second-wash were combined, and the volume was adjusted to 45 ml with sodium chloride solution (0.9%). The mixture was centrifuged at 1500 rpm for 5-7 minutes at 4° C., and the supernatant discarded. Ten percent cRBC solution was prepared by resuspending the final pellet in ten times the original volume.

6.3 Determination of Hemaglutinin (HA) Content in Concentrated Influenza Viral Lysate Stocks

In order to perform an HA Inhibition Assay, the HA titer of the viral lysate stock must be determined. The HA Inhibition Assay requires a viral lysate screening stock at a concentration of 8HA per 50 μl of solution. Determination of the viral lysate HA titer allows for proper dilution of the viral lysate stock for the HA Inhibition Assay.

Fresh 0.5% cRBC reagent was prepared daily. Sodium chloride solution (0.9%, 50 μl) was distributed into the wells of a Falcon® Non-Tissue Culture Treated Plate, 96 well, U-Bottom with Low Evaporation Lid. Viral Lysate (100 μl) was distributed into a set of wells, which did not contain the sodium chloride solution. Half of the viral lysate (50 μl) was then transferred into the next well (containing 50 μl sodium chloride), creating a 1:2 dilution. This serial dilution for both replicates was continued through the last well containing the sodium chloride. cRBC solution (0.5%, 50 μl) was disctributed into the wells. Wells with no viral lysate served as negative controls. The assay was allowed to incubate for 45 minutes at room temperature, ensuring that the plate is not jostled.

If there is too little viral lysate in the dilution to ensure hemagglutination, the cRBC's in the well settle at the bottom of the well due to gravity. Any well containing partial or total settling of the cRBC's to the bottom of the well is negative. The last well with complete suspension of the cRBC's in the solution was determined for the HA titer of the viral lysate.

6.4 Titration of the Influenza Antigen Working Stock to Verify HA Content

Prior to performing the HA Inhibition Assay, the HA titer of the viral lysate working stock must be validated. The working stock should be 8HA per 50 μl. Fresh 0.5% cRBC reagent was prepared daily. Predetermined dilution of the viral lysate to yield the presumptive 8 HA working stock was performed. Dilutions were prepared with sodium chloride solution (0.9%).

Sodium chloride solution (0.9%, 50 μl) was distribted into the wells of a Falcon® Non-Tissue Culture Treated Plate, 96 well, U-Bottom with Low Evaporation Lid. The presumptive 8HA/50 μl working stock (100 μl) was distributed into a single row or column of “start wells.” Half volume (50 μl) of the stock was transferred from the start well to a second well, creating a 1:2 dilution. Using the 1:2 dilution, repeat the process and continue until the dilution series was complete. A complete dilution set had wells containing 0.0625 HA to 8HA. cRBC reagent (0.5%, 50 μl) was distributed into each well containing some level of HA, and the assay was allowed to incubate for 45 minutes at room temperature, ensuring that the plate is not jostled.

If too little viral lysate HA in the dilution to ensure hemagglutination, the cRBC's in the well settle at the bottom of the well due to gravity. Any well containing partial or total settling of the cRBC's to the bottom of the well is negative. The last well with complete suspension of the cRBC's in the solution is the HA titer of the viral lysate stock. If the stock was truly an 8HA per 50 μl stock, then upon retitration, the last positive wells contained 1HA.

6.5 Mesurement of HA Specific Antibody Titer by HAI

Inoculums were administered intramusclularly (Brown Norway Rats) or intradermally (Balb/c Mice and Hartley Guinea Pigs), and sera from the subject were collected and used as test samples. Fresh cRBC reagent was prepared daily. Sodium Chloride solution (0.9%) was added to wells of a Falcon® Non-Tissue Culture Treated Plate, 96 well, U-Bottom with Low Evaporation Lid. Viral lysate stock (8 HA/50 μl) was added to wells. Appropriate volume of test serum was added to a single row or column of “start wells,” and a serial dilution was performed by transferring 50 μl of the serum dilution from the “start wells” into the next well, creating a 1:2 dilution. When completed, wells contained a serial serum dilution and a constant amount of viral lysate antigen, being 4HA per well. cRBC reagent (0.5%, 50 μl) was added to each well, including negative control wells, which contained no HA. The assay was allowed to incubate for 45 minutes at room temperature, ensuring that the plate is not jostled. For determination, plates were tilted at a 70-degree angle for 5 minutes, and viewed on a light box.

6.6 Results

6.6.1 Lutrol and Methylcellulose

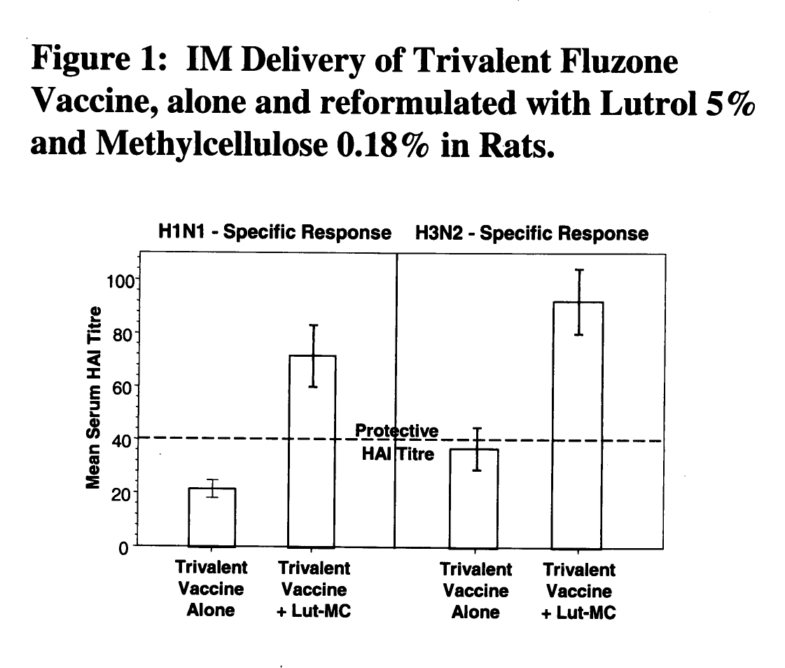

Rats (n=10 per group) were immunized intramuscularly with trivalent Fluzone® vaccine alone or reformulated with 5% lutrol and 0.18% methylcellulose. Sera were collected on d21 and screened for antibodies specific to the H1N1 New Caledonia strain or the H3N2 Panama strain by HAI assay. As shown in FIG. 1, the vaccine reformulated with lutrol-methylcellulose elicited higher immune responses than those obtained from Fluzone® vaccine alone with regard to both the New Caledonia and Panama strains.

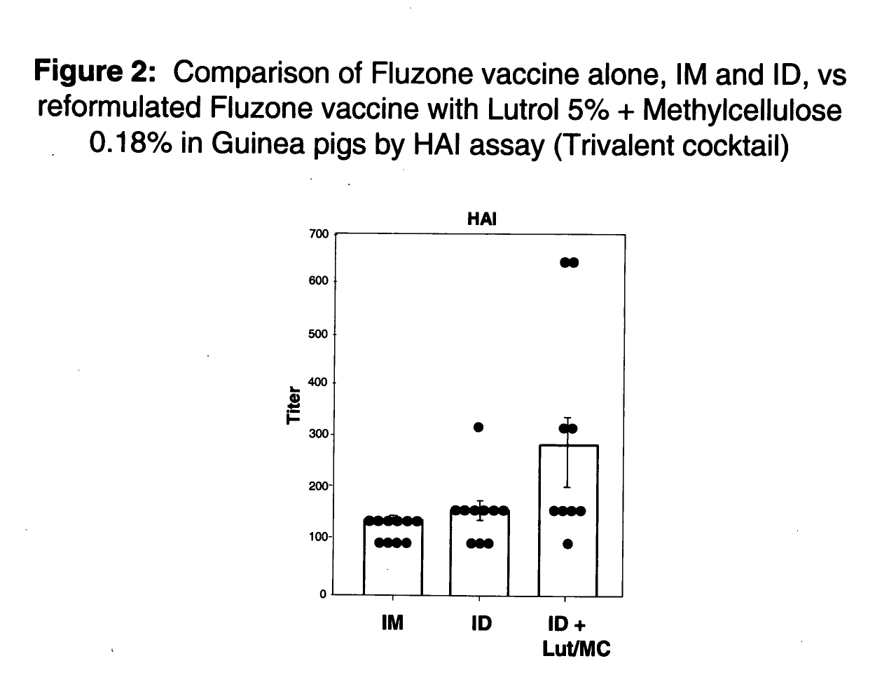

In another experiment, guinea pigs (n=10 per group) were immunized with trivalent Fluzone® vaccine alone intramuscularly or intradermally or intradermally with Fluzone® vaccine reformulated with 5% lutrol and 0.18% methylcellulose. Sera were collected on d21 and screened against a cocktail consisting of H1N1 New Caledonia strain, the H3N2 Panama strain and the Hong Kong B strain by HAI assay. As shown in FIG. 2, the ID administration of the vaccine reformulated with lutrol and methylcellulose elicited a higher immune response than Fluzone® vaccine alone. These results clearly show that the combination of lutrol and methylcellulose exhibits an adjuvant activity when administered to a subject together with an immunogen.

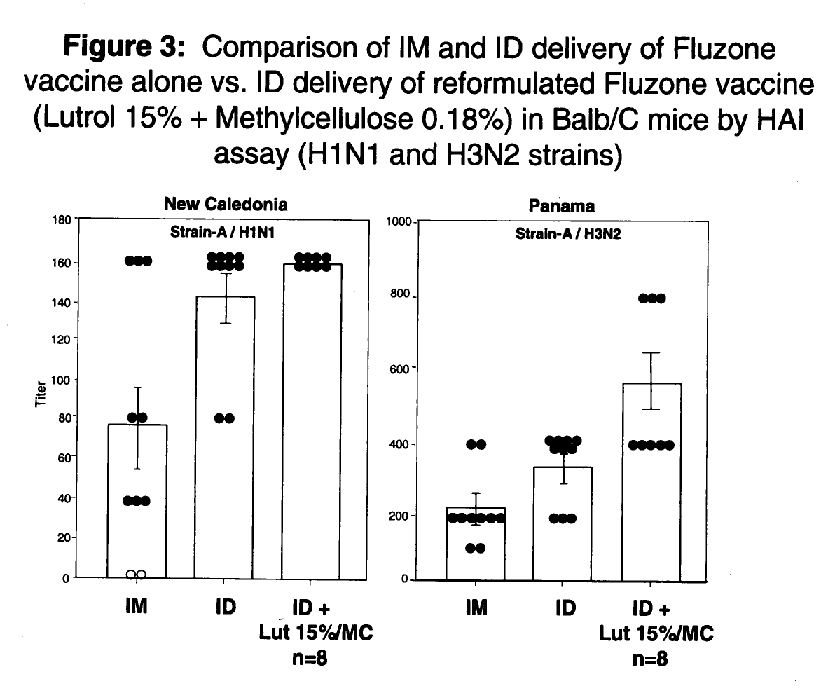

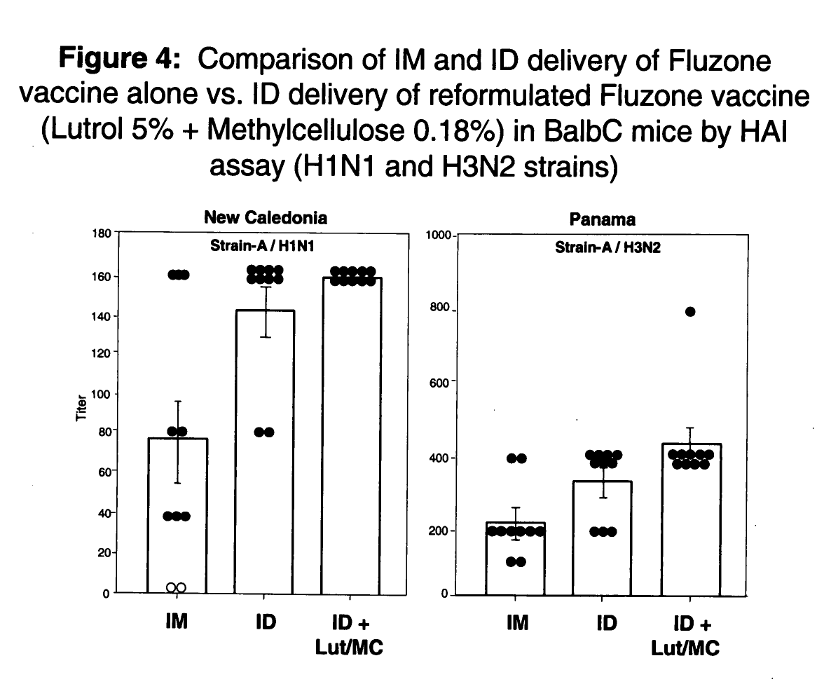

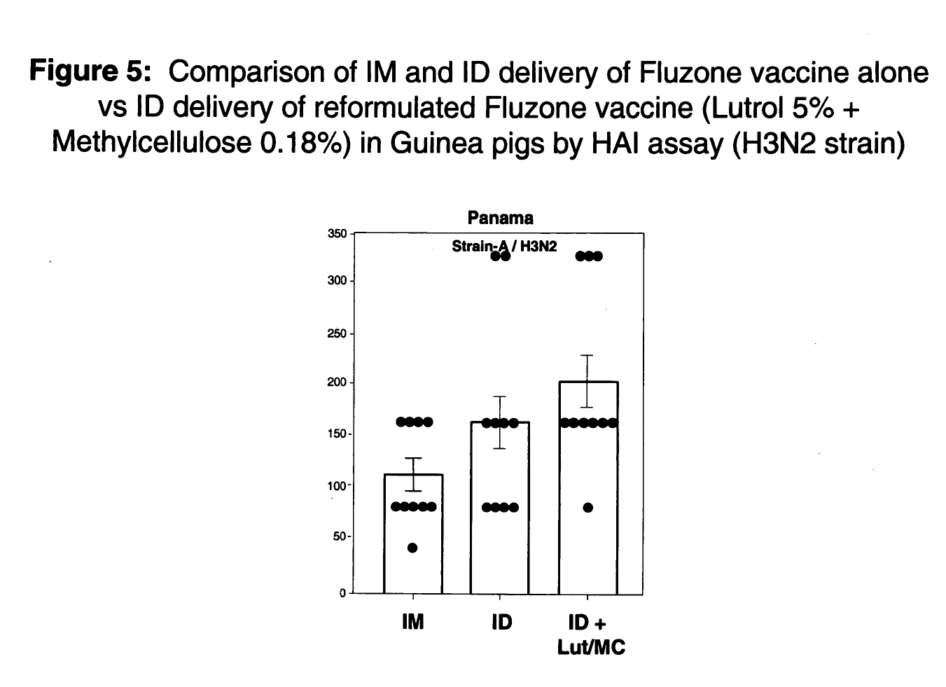

The adjuvant activity exhibited by the combination of lutrol and methylcelluose was also apparent regardless of whether they were administered to Balb/c mice (FIGS. 3 and 4) or guinea pigs (FIG. 5). In addition, the combination of lutrol and methylcellulose exhibited adjuvant activity in broad ranges of lutrol concentration, in particular, where the concentration of lutrol was 15% (FIG. 3) or 5% (FIGS. 4-5).

6.6.2 Lutrol and Urea

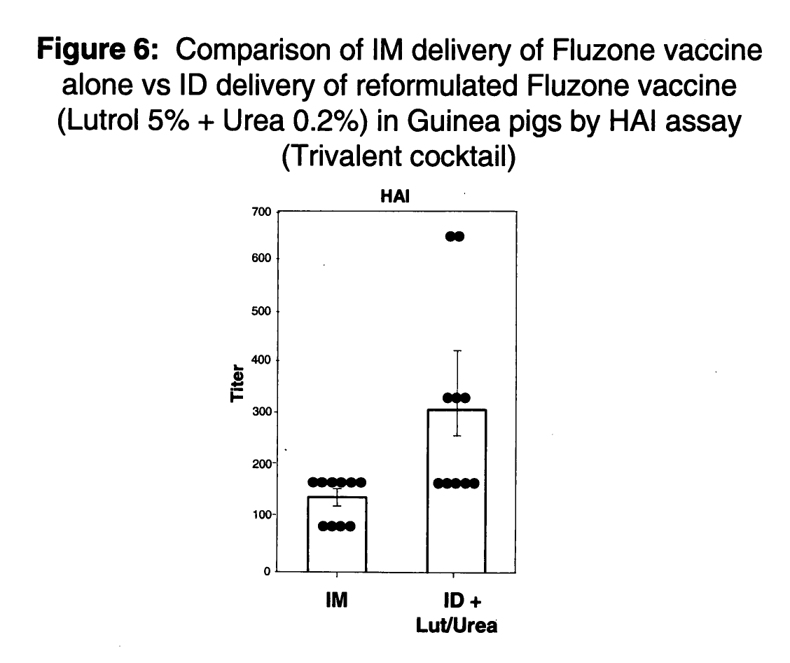

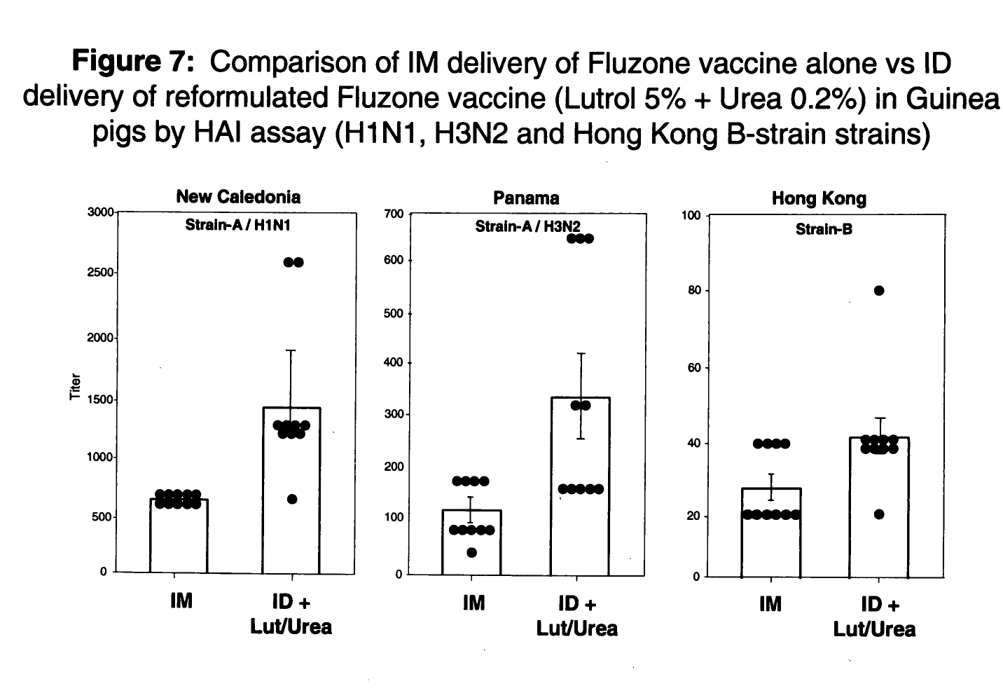

Guinea pigs (n=10 per group) were immunized with trivalent Fluzone® vaccine alone intramuscularly or intradermally with Fluzone® vaccine reformulated with 5% lutrol and 0.2% urea. Sera were collected on d21 and screened against a cocktail consisting of H1N1 New Caledonia strain, the H3N2 Panama strain and the Hong Kong B strain by HAI assay (FIG. 6) and against each of the individual strains, H1N1 New Caledonia, H3N2 Panama strain or Hong Kong B strain by HAI assay (FIG. 7). Data indicate that the Fluzone® vaccine reformulated with lutrol and urea elicited a higher immune response than the Fluzone® vaccine alone. These results clearly show that the combination of lutrol and urea exhibits an adjuvant activity when administered to a subject together with an immunogen.

6.6.3 Gelatin and Methylcellulose

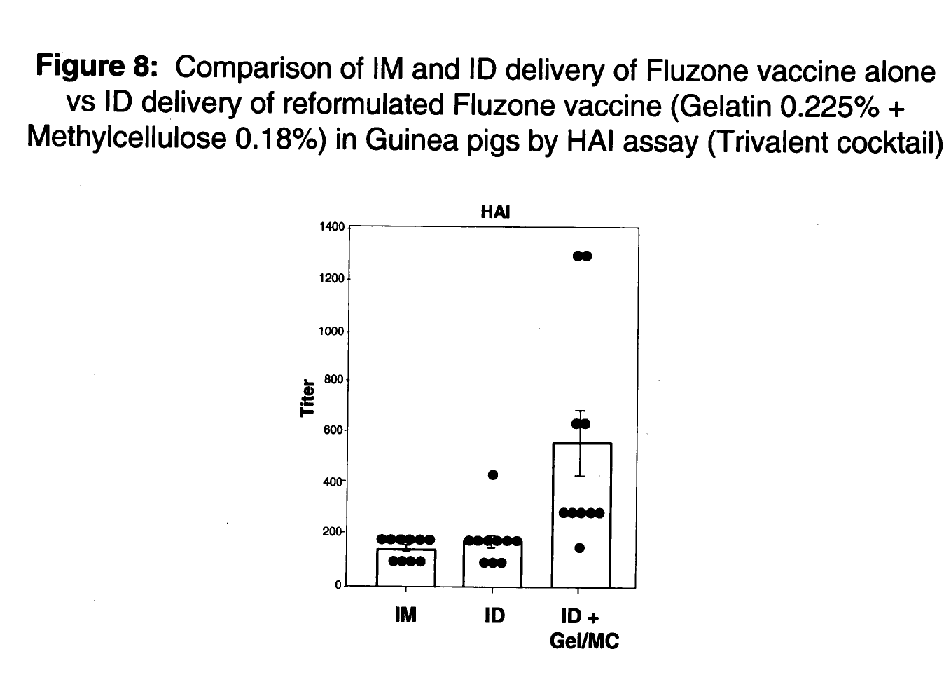

In another experiment, guinea pigs (n=10 per group) were immunized with trivalent Fluzone® vaccine alone intramuscularly or intradermally or intradermally with Fluzone® vaccine reformulated with 0.225% gelatin and 0.18% methylcellulose. Sera were collected on d21 and screened against a cocktail consisting of H1N1 New Caledonia strain, the H3N2 Panama strain and the Hong Kong B strain by HAI assay. As shown in FIG. 8, the Fluzone® vaccine reformulated with gelatin and methylcellulose elicited a higher immune response than the Fluzone® vaccine alone. The results show that the combination of gelatin and methylcellulose exhibits an adjuvant activity when administered to a subject together with an immunogen.

6.6.4 Lutrol and Sorbitol

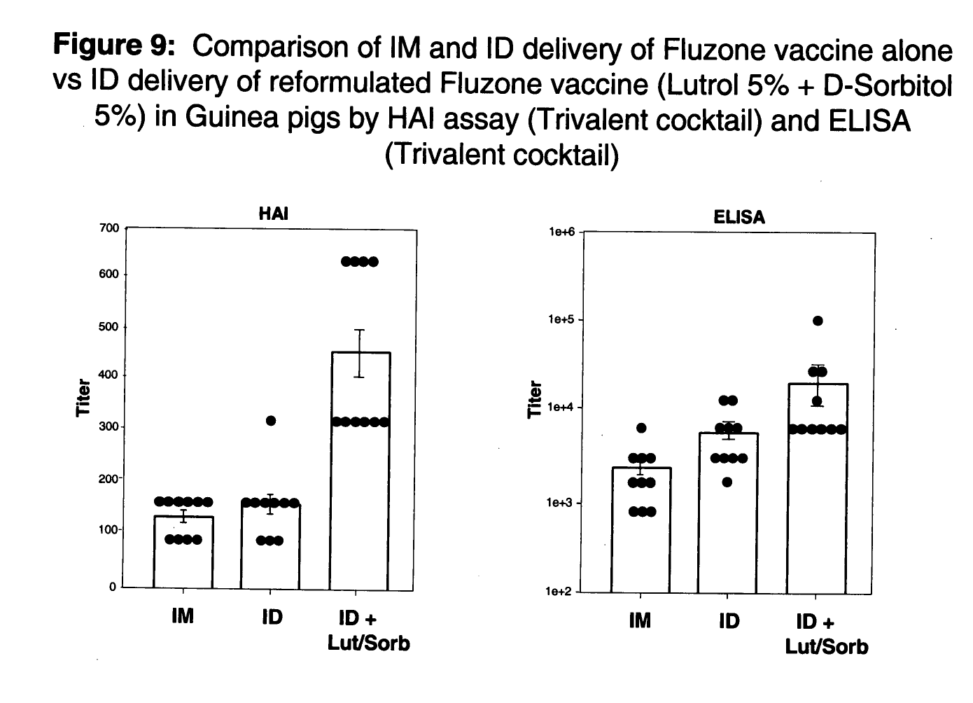

In yet another experiment, guinea pigs (n=10 per group) were immunized with trivalent Fluzone® vaccine alone intramuscularly or intradermally or intradermally with Fluzone® vaccine reformulated with 5% Lutrol and 5% D-Sorbitol. Sera were collected on d21 and screened against a cocktail consisting of H1N1 New Caledonia strain, the H3N2 Panama strain and the Hong Kong B strain by HAI assay. As shown in FIG. 9, the Fluzone® vaccine reformulated with lutrol and sorbitol elicited a higher immune response than the Fluzone® vaccine alone, as determined by HAI assay. The results show that the combination of lutrol and sorbitol exhibits an adjuvant activity, when administered to a subject together with an immunogen.

6.7 Draize Scoring of the Excipients Combinations

To assess the skin irritation that may be caused by the combination of excipients used in the compositions of the invention, Draize scoring tests were performed following the administration of certain excipients combinations to either Yorkshire swine or Hartley guinea pigs. A typical scoring scales are shown in Table 1 below.

TABLE 1
Draize Scoring
Key to interpreting skin reactions - Draize Scoring

| | |
|---|---|
| Erythema Score | Edema Score |

| | | | |
|---|---|---|---|
| No erythema | 0 | No edema | 0 |
| Slight erythema | 1 | Slight edema | 1 |
| (barely perceptible) | | (barely perceptible) | |
| Well-defined erythema | 2 | Well-defined edema | 2 |
| Moderate to severe | 3 | Moderate to severe | 3 |
| Severe erythema (beet | 4 | Sever edema (extending | 4 |
| redness to administration | | beyond the site | |
| sight, injury by depth | | | |

Erythema Draize scores of various combinations were as follows:

TABLE 2
Lutrol (10%) and Urea (5%), 200 μl per Injection:
Combination was delivered without vaccine to swine
using 31 guage 1.0 mm, 1.5 mm, or 2.0 mm needles

| Needle | 1 Hour After Injection | | | 24 Hours After Injection | | |
|---|---|---|---|---|---|---|
| 1.0 mm | 1 | 1+ | 1 | 1 | 1 | 1 |
| 1.5 mm | 0 | 1 | 1+ | 1 | 0 | 1+ |
| 2.0 mm | 0 | 0 | 0 | 0 | 0 | 0 |

TABLE 3
Various Combinations, 50 μl per Injection: Specified
combinations were delivered without vaccine to guinea
pigs using 34 guage, 1.0 mm needles

| | Immediately After | | 1 Hour After | | 24 Hours After | |
|---|---|---|---|---|---|---|
| Combinations | Injection | | Injection | | Injection | |
| Lutrol (5%) + | 1 | 1 | 1 | 1 | 1+ | 1 |
| methylcellulose | | | | | | |
| (0.18%) | | | | | | |
| Lutrol (5%) + | 1 | 1 | 0 | 0 | 0 | 0 |
| Urea (0.2%) | | | | | | |
| Lutrol (5%) + | 1 | 1 | 0 | 0 | 1+ | 1+ |
| Sorbitol (5%) | | | | | | |
| Gelatin (0.225%) + | 1 | 1 | 0 | 0 | 1 | 0 |
| methylcellulose | | | | | | |
| (0.18%) | | | | | | |

TABLE 4
Various Combinations, 200 μl per Injection:
Specified combinations were delivered without vaccine
to swine using 34 guage, 1.5 mm needles

| Combinations | 1 Hour After Injection | | | 24 Hours After Injection | | |
|---|---|---|---|---|---|---|
| Lutrol (5%) + | 2 | 2 | 2 | 0 | 0 | 0 |
| Methylcellulose | | | | | | |
| (0.18%) | | | | | | |
| Lutrol (5%) + | 1 | 2 | 1+ | 0 | 0 | 0 |
| Urea (0.2%) | | | | | | |
| Lutrol (5%) + | 0 | 0 | 1+ | 0 | 0 | 0 |
| Sorbitol (5%) | | | | | | |
| Gelatin (0.225%) + | 1 | 1 | 1 | 0 | 0 | 0 |
| Methylcellulose | | | | | | |
| (0.18%) | | | | | | |
| Lutrol (5%) + | 0 | 0 | 0 | 0 | 1 | 1+ |
| Gelatin (0.225%) | | | | | | |

As shown in Tables 2-4, none of the combinations tested exhibited a serious skin irritation when administered to a subject. The results suggest that the excipients combinations of the invention are also safe for the use in patients.

The invention described and claimed herein is not to be limited in scope by the specific embodiments herein disclosed since these embodiments are intended as illustration of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

Throughout this application various publications are cited. Their contents are hereby incorporated by reference into the present application in their entireties for all purposes.

What is claimed is:

1. An immunogenic composition comprising an immunogen and a combination of lutrol and methylcellulose.

2. The immunogenic composition of claim 1, wherein the composition is a vaccine.

3. The immunogenic composition of claim 2, wherein the vaccine is an influenza vaccine.

4. The immunogenic composition of claim 1, wherein the concentration of lutrol used in the composition is from about 1% w/v to about 25% w/v of the composition.

5. The immunogenic composition of claim 4, wherein the concentration of lutrol used in the composition is from about 3% w/v to about 15% w/v of the composition.

6. The immunogenic composition of claim 5, wherein the concentration of lutrol used in the composition is from about 5% w/v to about 10% w/v of the composition.

7. The immunogenic composition of claim 1, wherein the concentration of methylcellulose used in the composition is from about 0.001% w/v to about 1% w/v of the composition.

8. The immunogenic composition of claim 7, wherein the concentration of methylcellulose used in the composition is from about 0.01% w/v to about 0.5% w/v of the composition.

9. The immunogenic composition of claim 8, wherein the concentration of methylcellulose used in the composition is from about 0.02% w/v to about 0.1% w/v of the composition.

10. The immunogenic composition of claim 1, wherein the composition is suitable for intradermal, epidermal, intramuscular, transdermal, junctional, nasal, or subcutaneous administration.

11. An immunogenic composition comprising an immunogen and a combination of lutrol and sorbitol.

12. The immunogenic composition of claim 11, wherein the composition is a vaccine.

13. The immunogenic composition of claim 12, wherein the vaccine is an influenza vaccine.

14. The immunogenic composition of claim 11, wherein the concentration of lutrol used in the composition is from about 1% w/v to about 25% w/v of the composition.

15. The immunogenic composition of claim 14, wherein the concentration of lutrol used in the composition is from about 3% w/v to about 15% w/v of the composition.

16. The immunogenic composition of claim 15, wherein the concentration of lutrol used in the composition is from about 5% w/v to about 10% w/v of the composition.

17. The immunogenic composition of claim 11, wherein the concentration of sorbitol used in the composition is from about 0.5% w/v to about 25% w/v of the composition.

18. The immunogenic composition of claim 17, wherein the concentration of sorbitol used in the composition is from about 3% w/v to about 15% w/v of the composition.

19. The immunogenic composition of claim 18, wherein the concentration of sorbitol used in the composition is from about 5% w/v to about 10% w/v of the composition.

20. The immunogenic composition of claim 11, wherein the composition is suitable for intradermal, epidermal, intramuscular, transdermal, junctional, nasal, or subcutaneous administration.

21. An immunogenic composition comprising an immunogen and a combination of lutrol and urea.

22. The immunogenic composition of claim 21, wherein the composition is a vaccine.

23. The immunogenic composition of claim 22, wherein the vaccine is an influenza vaccine.

24. The immunogenic composition of claim 21, wherein the concentration of lutrol used in the composition is from about 1% w/v to about 25% w/v of the composition.

25. The immunogenic composition of claim 24, wherein the concentration of lutrol used in the composition is from about 3% w/v to about 15% w/v of the composition.

26. The immunogenic composition of claim 25, wherein the concentration of lutrol used in the composition is from about 5% w/v to about 10% w/v of the composition.

27. The immunogenic composition of claim 21, wherein the concentration of urea used in the composition is from about 0.01% w/v to about 40% w/v of the composition.

28. The immunogenic composition of claim 27, wherein the concentration of urea used in the composition is from about 0.1% w/v to about 10% w/v of the composition.

29. The immunogenic composition of claim 28, wherein the concentration of urea used in the composition is from about 0.2% w/v to about 1% w/v of the composition.

30. The immunogenic composition of claim 21, wherein the composition is suitable for intradermal, epidermal, intramuscular, transdermal, junctional, nasal, or subcutaneous administration.

31. An immunogenic composition comprising an immunogen and a combination of lutrol and chitosan.

32. The immunogenic composition of claim 31, wherein the composition is a vaccine.

33. The immunogenic composition of claim 32, wherein the vaccine is an influenza vaccine.

34. The immunogenic composition of claim 31, wherein the concentration of lutrol used in the composition is from about 1% w/v to about 25% w/v of the composition.

35. The immunogenic composition of claim 34, wherein the concentration of lutrol used in the composition is from about 3% w/v to about 15% w/v of the composition.

36. The immunogenic composition of claim 35, wherein the concentration of lutrol used in the composition is from about 5% w/v to about 10% w/v of the composition.

37. The immunogenic composition of claim 31, wherein the concentration of chitosan used in the composition is from about 0.01% w/v to about 1% w/v of the composition.

38. The immunogenic composition of claim 37, wherein the concentration of chitosan used in the composition is from about 0.05% w/v to about 0.5% w/v of the composition.

39. The immunogenic composition of claim 38, wherein the concentration of chitosan used in the composition is from about 0.1% w/v to about 0.25% w/v of the composition.

40. The immunogenic composition of claim 31, wherein the composition is suitable for intradermal, epidermal, intramuscular, transdermal, junctional, nasal, or subcutaneous administration.

41. An immunogenic composition comprising an immunogen and a combination of methylcellulose and gelatin.

42. The immunogenic composition of claim 41, wherein the composition is a vaccine.

43. The immunogenic composition of claim 42, wherein the vaccine is an influenza vaccine.

44. The immunogenic composition of claim 41, wherein the concentration of methylcellulose used in the composition is from about 0.001% w/v to about 1% w/v of the composition.

45. The immunogenic composition of claim 44, wherein the concentration of methylcellulose used in the composition is from about 0.01% w/v to about 0.5% w/v of the composition.

46. The immunogenic composition of claim 45, wherein the concentration of methylcellulose used in the composition is from about 0.02% w/v to about 0.1% w/v of the composition.

47. The immunogenic composition of claim 41, wherein the concentration of gelatin used in the composition is from about from about 0.01% w/v to about 5% w/v of the composition.

48. The immunogenic composition of claim 47, wherein the concentration of gelatin used in the composition is from about 0.05% w/v to about 0.5% w/v of the composition.

49. The immunogenic composition of claim 48, wherein the concentration of gelatin used in the composition is from about 0.1% w/v to about 0.225% w/v of the composition.

50. The immunogenic composition of claim 41, wherein the composition is suitable for intradermal, epidermal, intramuscular, transdermal, junctional, nasal, or subcutaneous administration.

51. An immunogenic composition comprising an immunogen and a combination of lutrol and gelatin.

52. The immunogenic composition of claim 51, wherein the composition is a vaccine.

53. The immunogenic composition of claim 52, wherein the vaccine is an influenza vaccine.

54. The immunogenic composition of claim 51, wherein the concentration of lutrol used in the composition is from about 1% w/v to about 25% w/v of the composition.

55. The immunogenic composition of claim 54, wherein the concentration of lutrol used in the composition is from about 3% w/v to about 15% w/v of the composition.

56. The immunogenic composition of claim 55, wherein the concentration of lutrol used in the composition is from about 5% w/v to about 10% w/v of the composition.

57. The immunogenic composition of claim 51, wherein the concentration of gelatin used in the composition is from about 0.01% w/v to about 5% w/v of the composition.

58. The immunogenic composition of claim 57, wherein the concentration of gelatin used in the composition is from about 0.05% w/v to about 0.5% w/v of the composition.

59. The immunogenic composition of claim 58, wherein the concentration of gelatin used in the composition is from about 0.1% w/v to about 0.225% w/v of the composition.

60. The immunogenic composition of claim 51, wherein the composition is suitable for intradermal, epidermal, intramuscular, transdermal, junctional, nasal, or subcutaneous administration.
